(19) United States
(12) Patent Application Publication
(10) Pub. No.: US 2025/0189528 A1
(43) Pub. Date: Jun. 12, 2025

(54) BIOMARKERS FOR DISEASE PROGRESSION AND/OR RECURRENCE IN SQUAMOUS CELL CARCINOMA

(71) Applicant: AMLO Biosciences Limited, Newcastle Upon Tyne (GB)

(72) Inventors: Penny LOVAT, Newcastle Upon Tyne (GB); Michael ALEXANDER, Newcastle Upon Tyne (GB); Niki STEFANOS, Newcastle Upon Tyne (GB)

(21) Appl. No.: 18/840,803

(22) PCT Filed: Feb. 6, 2023
(86) PCT No.: PCT/GB2023/050252

§ 371 Date: Aug. 22, 2024

(30) Foreign Application Priority Data
Feb. 24, 2022 (GB) 2202569.6

(51) Int. Cl. G01N 33/574; A61K 45/00

(52) U.S. Cl. CPC G01N 33/5743; A61K 45/00

(57) ABSTRACT

The invention relates to methods for determining whether a subject has an increased risk of progression and/or recurrence of squamous cell carcinoma (SCC). In certain embodiments, the invention relates to determining the expression of Ambra-1 and p62 in one or more tissue samples obtained from the subject, wherein the tissue sample(s) comprise a primary tumour and tissue overlying the primary tumour and a decrease or loss in the expression of Ambra-1 and a decrease or loss in the expression of p62 in the tissue sample(s) compared to one or more reference level(s) is indicative of an increased risk of progression and/or recurrence of the tumour. The invention further relates to methods of treating subjects identified to be at increased risk of having progressive and/or recurring SCC.

### FIELD OF THE INVENTION

The present invention relates inter alia to methods for determining whether a subject has an increased risk of progression and/or recurrence of squamous cell carcinoma (SCC). In certain embodiments, the invention relates to determining the expression of Ambra-1 and p62 in one or more tissue samples obtained from the subject, wherein the tissue sample(s) comprise a primary tumour and tissue overlying the primary tumour and a decrease or loss in the expression of Ambra-1 and a decrease or loss in the expression of p62 in the tissue sample(s) compared to one or more reference level(s) is indicative of an increased risk of progression and/or recurrence of the tumour. The invention also relates to methods of treating subjects identified to be at increased risk of having progressive and/or recurring SCC.

### BACKGROUND TO THE INVENTION

SCC (also known as epidermoid carcinoma) comprises a range of cancer types that result from squamous cells. These cells form the surface of skin, the lining of hollow organs in the body and the lining of the respiratory and digestive tracts.

SCC comprises one of the largest subsets of cancer. For example, the global incidence of cutaneous squamous cell carcinoma (cSCC) is increasing worldwide and is a considerable health care burden. Surgical excision of the primary tumour has a high likelihood of being a curable event. The prognosis, however, for patients who develop advanced disease is poor. Coupled with the inability of current clinical parameters to accurately predict metastatic risk (Weinberg et al., 2007) there is hence an urgent unmet need for the development of reliable and consistent biomarkers able to identity subsets of patients with cSCC at risk of disease recurrence or metastasis.

As with many cancers, prognosis and survival for SCC is highly dependent on early diagnosis and treatment. Current prognosis is based on histological features of the primary tumour in line with the most recent TNM system of the International Union Against Cancer (UICC), 2009 and the American Joint Committee on Cancer (AJCC) 2018. These systems are not optimal since they have been developed for all cases of SCCs with very different aggressiveness. Consequently, 16% of patients judged to have a low-risk prognosis under this system will experience tumour recurrence, metastasis and/or death. As prognostic risk cannot be accurately determined at an individual level, the default is to adopt intensive and costly follow-up schedules for all patients. However, for 84% of low-risk patients these intensive and costly follow-up schedules are unnecessary.

Although the AJCC staging system can predict outcomes in the majority of patients, there is still no accurate indicator to predict those individual patients with early-stage disease at diagnosis whose tumours will progress and/or reoccur. Thus, there remains a need for tests which accurately prognose an individual's risk of disease recurrence and spread at the point of initial diagnosis.

The focus of current research is towards refinement of high-risk histological features of the primary tumour (Hirshoren N et al 2017) or identification of tumour genetic markers linked to poor prognosis (Qi R et al 2018, Chen R et al 2018). There are challenges with both approaches, especially in the case of genetic tests due to the high background level of mutational burden in UV-exposed skin, the need for specialist equipment and analysis packages and in most cases, the need for a significant amount of tissue per test.

There remains a need for identification of prognostic markers that can accurately identify those patients with early-stage SCC at high risk of tumour progression and/or recurrence.

It is an aim of certain embodiments of the present invention to at least party mitigate the above-mentioned problems associated with the prior art.

### Summary of Certain Embodiments of the Invention

The present invention relates to analysis of a cohort of non and metastatic cSCC samples. Unexpectedly, the inventors have found that a decrease or loss of Ambra-1 in the SCC tumour and a decrease or loss of p62 in peritumoural epidermis is indicative of a poor prognosis. Surprisingly, the combination of a decrease or loss of Ambra-1 (e.g., a decrease or loss of cytoplasmic Ambra-1 in the tumour growth front) and a decrease or loss of p62 (e.g., a decrease or loss of cytoplasmic p62 in the peritumoral epidermis) is associated with an increased risk of cSCC progression and/or recurrence.

Thus, certain aspects of the invention provide immunohistochemical (IHC) tests based on the combined detection of Ambra-1 and p62 in tissue samples comprising a primary tumour and/or tissue overlying the primary tumour obtained from subjects with SCC.

The methods of certain embodiments of the invention may significantly improve on the existing state of the art by providing accurate risk prediction based on tumour biology rather than an estimated risk based on generic histological risk factors. Accurate prognosis of SCC improves subject outcome and reduces follow-up costs for healthcare providers.

Accordingly, an aspect of the invention provides a method of determining whether a subject has an increased risk of progression and/or recurrence of SCC, the method comprising determining the expression of Ambra-1 and p62 in one or more SCC tissue samples obtained from the subject, wherein the tissue sample(s) comprise a primary tumour and tissue overlying the primary tumour, and wherein a decrease or loss in the expression of Ambra-1 and a decrease or loss in the p62 in the SCC tissue sample(s) compared to one or more reference level(s) is indicative of an increased risk of progression and/or recurrence of the SCC.

Typically, expression of Ambra-1 and/or p62 is determined in the cytoplasm of cells within the SCC tissue biopsy.

Typically, expression of Ambra-1 (e.g., cytoplasmic Ambra-1) is determined in the primary tumour. Preferably, expression of Ambra-1 (e.g., cytoplasmic Ambra-1) is determined in the tumour growth front of the primary tumour.

Typically, expression of p62 (e.g., cytoplasmic p62) is determined in the tissue overlying the primary tumour. In an embodiment, expression of p62 (e.g., cytoplasmic p62) is determined in the peritumoral epidermis overlying the primary tumour.

In an embodiment, expression of cytoplasmic Ambra-1 is determined in the tumour growth front of the primary tumour and expression of cytoplasmic p62 is determined in the peritumoral epidermis overlying the primary tumour.

Typically, the primary tumour is moderately/poorly differentiated.

Typically, the subject has cutaneous SCC (cSCC).

In a further aspect, the invention also provides a method of determining a treatment regime for a subject suffering from SCC, the method comprising:
(a) determining whether the subject has an increased risk of progression and/or recurrence of a primary tumour according to any method described herein; and
(b) if expression of Ambra-1 and p62 is decreased or lost in the SCC tissue sample(s) compared to the one or more reference level(s), treating the subject with a systemic anti-cancer treatment regime.

### DETAILED DESCRIPTION OF CERTAIN EMBODIMENTS

Embodiments of the present invention will now be described hereinafter, by way of example only, with reference to the accompanying drawings in which:

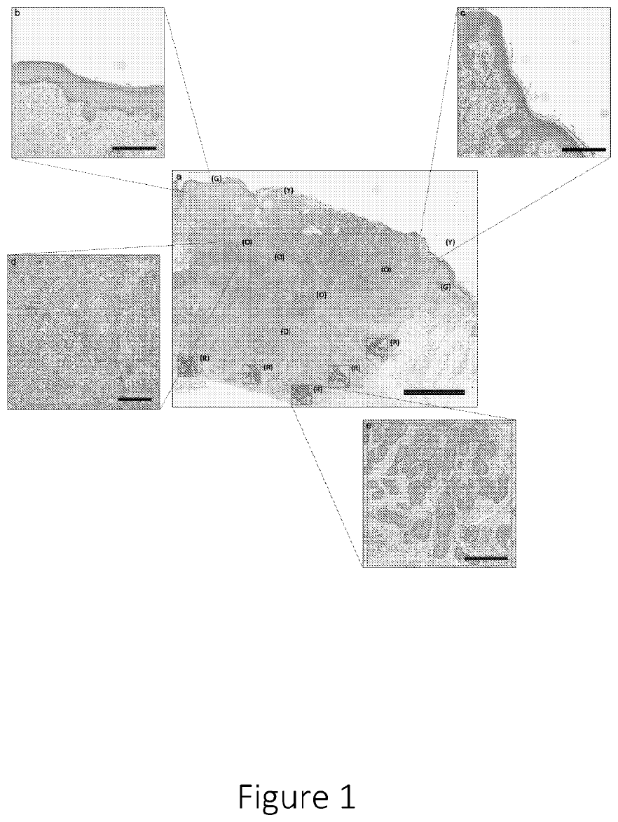

FIG. 1 shows annotation methodology used to analyse expression of AMBRA1 and p62 in primary cSCC tumours. (a) Representative photomicrograph images of a cSCC tumour stained for AMBRA1 with completed annotations for the normal epidermis region (green (g)), peritumoural epidermis region (yellow (y)), tumour mass region (orange (o)) and tumour growth front region (red (r)). (b) Enlarged photomicrograph image of the normal epidermis annotated region of the cSCC tumour. (c) Enlarged photomicrograph image of the peritumoural epidermis annotated region of the cSCC tumour. (d) Enlarged photomicrograph image of the tumour mass annotated region of the cSCC tumour. (e) Enlarged photomicrograph image of the tumour growth front annotated region of the cSCC tumour. Visible staining was achieved via immunohistochemistry with a DAB counterstain. Image a was taken using bright field microscopy with a magnification of 0.8×. Scale bar=3 mm. Image b, d and e were taken using bright field microscopy with a magnification of 9.5×. Scale bar=300 μm. Image c was taken using bright field microscopy with a magnification of 10.3×. Scale bar=200 μm.

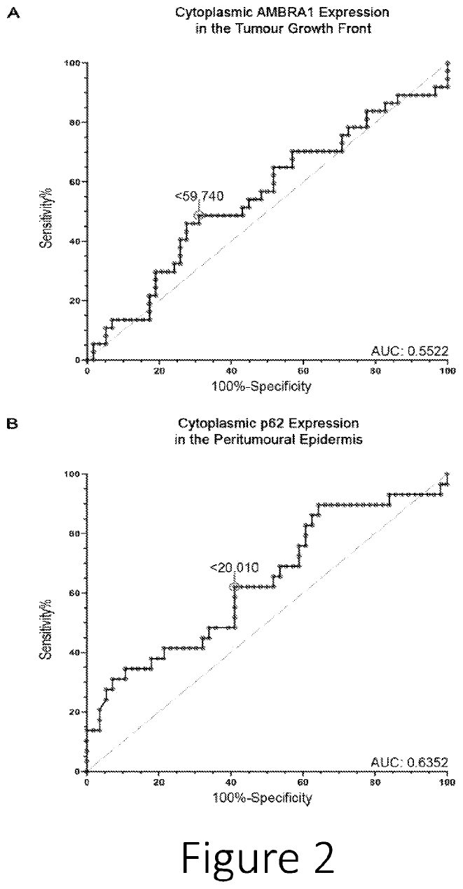

FIG. 2 shows loss of cytoplasmic AMBRA1 expression in the tumour growth front region and loss of cytoplasmic p62 expression in the peritumoural epidermis best predict cSCC recurrence or metastasis. (A) Receiver operating characteristic (ROC) curve for prediction of either cSCC reoccurrence or metastasis based on the cytoplasmic AMBRA1 H-score in the tumour growth front of all primary cSCC tumours (n=95). The AMBRA1 H-score with the highest specificity and sensitivity is highlighted by a red circle. AUC=area under the curve. (B) Receiver operating characteristic (ROC) curve for prediction of a cSCC event (reoccurrence/metastasis) based on the cytoplasmic p62 H-score in the peritumoural epidermis of all primary cSCC tumours (n=85). The p62 H-score with the highest specificity and sensitivity is highlighted by a red circle. AUC=area under the curve.

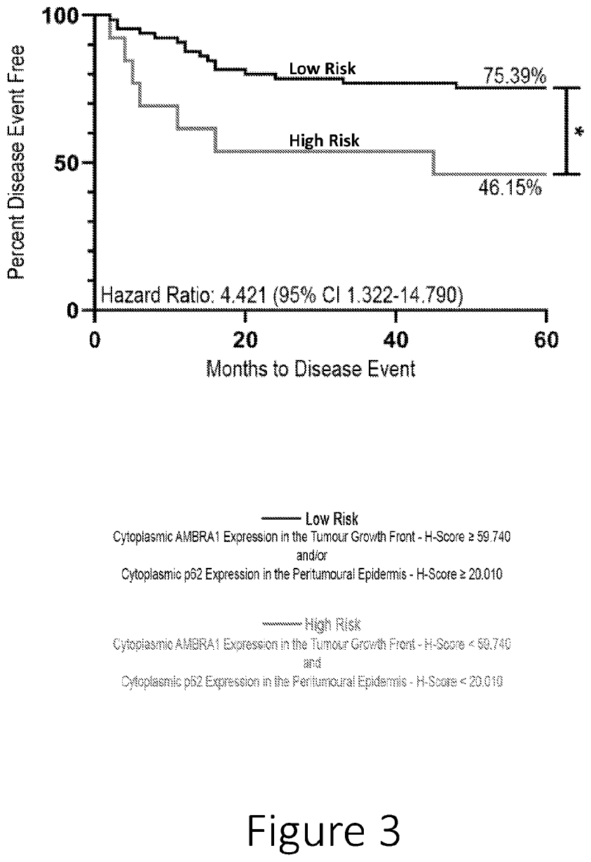

FIG. 3 shows cytoplasmic AMBRA1 expression in the tumour growth front region and cytoplasmic p62 expression in the peritumoural epidermis region act as a biomarker for cSCC patients. Kaplan-Meier survival analysis representing 60-month disease event free rate in 79 primary cSCC tumours stratified as low risk (n=65) and high risk (n=13) groups based on cytoplasmic AMBRA1 expression in the tumour growth front region and cytoplasmic p62 expression in the peritumoural epidermis. Statistics acquired by Mantel-Cox log-rank test and Mantel-Haenszel test (*P<0.05).

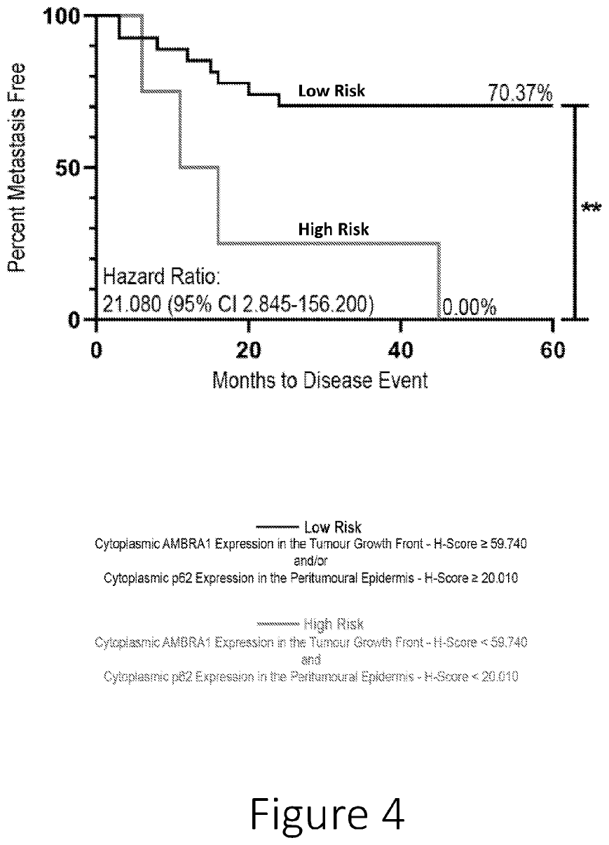

FIG. 4 shows Cytoplasmic AMBRA1 expression in the tumour growth front region and cytoplasmic p62 expression in the peritumoural epidermis region act as a prognostic biomarker for disease metastasis in moderately differentiated cSCC tumours. Kaplan-Meier survival analysis representing 60-month disease event free rate in 31 moderately-differentiated primary cSCC tumours stratified as low risk (n=27) and high risk (n=4) groups based on cytoplasmic AMBRA1 expression in the tumour growth front region and cytoplasmic p62 expression in the peritumoural epidermis. Statistics acquired by Mantel-Cox log-rank test and Mantel-Haenszel test (*P<0.05).

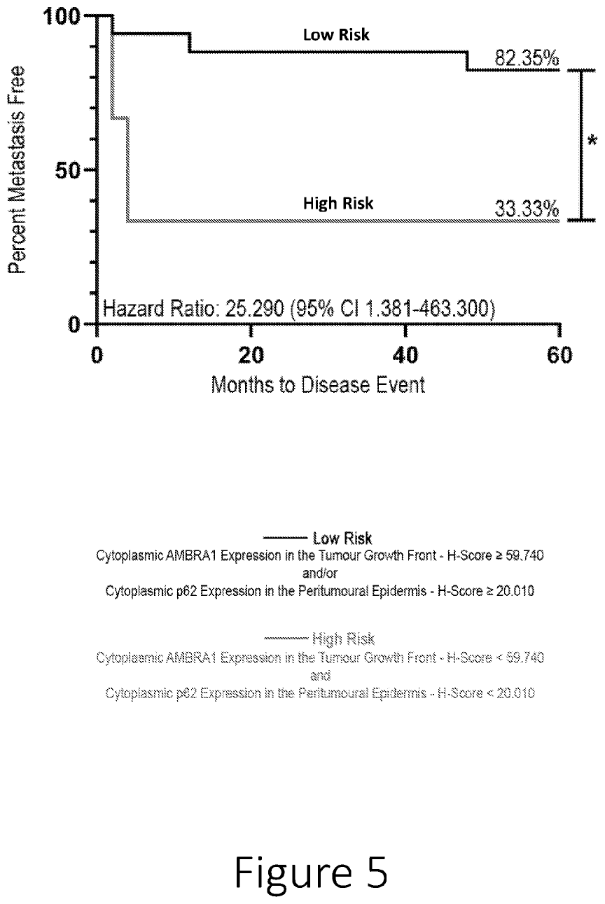

FIG. 5 shows cytoplasmic AMBRA1 expression in the tumour growth front region and cytoplasmic p62 expression in the peritumoural epidermis region act as a prognostic biomarker for disease metastasis in poorly differentiated cSCC tumours. Kaplan-Meier survival analysis representing 60-month disease event free rate in 20 poorly differentiated primary cSCC tumours stratified as low risk (n=17) and high risk (n=3) groups based on cytoplasmic AMBRA1 expression in the tumour growth front region and cytoplasmic p62 expression in the peritumoural epidermis. Statistics acquired by Mantel-Cox log-rank test and Mantel-Haenszel test (*P<0.05).

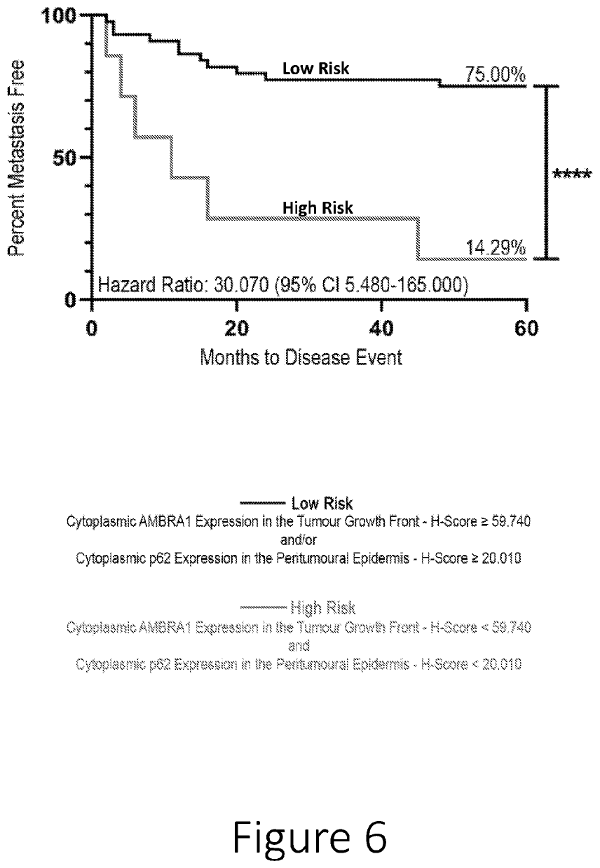

FIG. 6 shows cytoplasmic AMBRA1 expression in the tumour growth front region and cytoplasmic p62 expression in the peritumoural epidermis region act as a prognostic biomarker for disease metastasis in moderately/poorly differentiated cSCC tumours. Kaplan-Meier survival analysis representing 60-month disease event free rate in 51 primary cSCC tumours separated into low risk (n=44) and high risk (n=7) groups based on cytoplasmic AMBRA1 expression in the tumour growth front region and cytoplasmic p62 expression in the peritumoural epidermis. Statistics acquired by Mantel-Cox log-rank test and Mantel-Haenszel test (*P<0.05).

### SEQUENCE LISTING

SEQ ID NO: 1 shows the amino acid sequence of HCDR1 of the anti-Ambra-1 antibody
AbD33473
(SYWIH);
SEQ ID NO: 2 shows the amino acid sequence of HCDR2 of the anti-Ambra-1 antibody
AbD33473
(TIFPSRSYTTYSPSFQG);
SEQ ID NO: 3 shows the amino acid sequence of HCDR3 (DTPSTALKSPFDY) of the anti-
Ambra-1 antibody AbD33473;
SEQ ID NO: 4 shows the amino acid sequence of LCDR1 of the anti-Ambra-1 antibody
AbD33473
(SGSSSNIGYNYVY);
SEQ ID NO: 5 shows the amino acid sequence of LCDR2 of the anti-Ambra-1 antibody
AbD33473
(ENNKRPS);
SEQ ID NO: 6 shows the amino acid sequence of LCDR3 of the anti-Ambra-1 antibody
AbD33473
(SSWDSHSNSYV);
SEQ ID NO: 7 shows the amino acid sequence of HCFR1 of the anti-Ambra-1 antibody
AbD33473
(EVQLVQSGAEVKKPGESLKISCKGSGYSFS);
SEQ ID NO: 8 shows the amino acid sequence of HCFR2 of the anti-Ambra-1 antibody
AbD33473
(WVRQMPGKGLEWMG);
SEQ ID NO: 9 shows the amino acid sequence of HCFR3 of the anti-Ambra-1 antibody
AbD33473
(QVTISADKSISTAYLOWSSLKASDTAMYYCAR);
SEQ ID NO: 10 shows the amino acid sequence of HCFR4 of the anti-Ambra-1 antibody
AbD33473
(WGQGTLVTVSS);
SEQ ID NO: 11 shows the amino acid sequence of LCFR1 of the anti-Ambra-1 antibody
AbD33473
(DIVLTQPPSVSGAPGQRVTISC);
SEQ ID NO: 12 shows the amino acid sequence of LCFR2 of the anti-Ambra-1 antibody
AbD33473
(WYQQLPGTAPKLLIY);
SEQ ID NO: 13 shows the amino acid sequence of LCFR3 of the anti-Ambra-1 antibody
AbD33473
(GVPDRFSGSKSGTSASLAITGLQAEDEADYYC);
SEQ ID NO: 14 shows the amino acid sequence of LCFR4 of the anti-Ambra-1 antibody
AbD33473
(FGGGTKLTVLGQ);
SEQ ID NO: 15 shows the amino acid sequence of the VH domain of the anti-Ambra-1 antibody
AbD33473;
EVQLVQSGAEVKKPGESLKISCKGSGYSFSSYWIHWVRQMPGKGLEWMGTIFPSRSYTTYSPSFQGQV
TISADKSISTAYLQWSSLKASDTAMYYCARDTPSTALKSPFDYWGQGTLVTVSS
SEQ ID NO: 16 shows the amino acid sequence of the VL domain of the anti-Ambra-1 antibody
AbD33473;
DIVLTQPPSVSGAPGQRVTISCSGSSSNIGYNYVYWYQQLPGTAPKLLIYENNKRPSGVPDRFSGSKS
GTSASLAITGLQAEDEADYYCSSWDSHSNSYVFGGGTKLTVLGQ
SEQ ID NO: 17 shows the amino acid sequence of the Fd chain (VH domain and constant
domain) of the Fab region of the anti-Ambra-1 antibody AbD33473;
EVQLVQSGAEVKKPGESLKISCKGSGYSFSSYWIHWVRQMPGKGLEWMGTIFPSRSYTTYSPSFQGQV
TISADKSISTAYLQWSSLKASDTAMYYCARDTPSTALKSPFDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKS
SEQ ID NO: 18 shows the amino acid sequence of the light chain (VL domain and constant
domain) of the Fab region of the anti-Ambra-1 antibody AbD33473;
DIVLTQPPSVSGAPGQRVTISCSGSSSNIGYNYVYWYQQLPGTAPKLLIYENNKRPSGVPDRFSGSKS
GTSASLAITGLQAEDEADYYCSSWDSHSNSYVFGGGTKLTVLGQPKAAPSVTLFPPSSEELQANKATL
VCLISDFYPGAVTVAWKADSSPVKAGVETTTPSKQSNNKYAASSYLSLTPEQWKSHRSYSCQVTHEGS
TVEKTVAPTEA
SEQ ID NO: 19 shows the nucleic acid sequence encoding the Fd chain of the Fab region and
tags (alkaline phosphatase dimerization domain sequence (AP), FLAG tag (DYKDDDDK) and
His6 tag of the anti-Ambra-1 antibody AbD33473;
GAAGTGCAATTGGTGCAGAGCGGTGCGGAAGTGAAAAAACCGGGCGAAAGCCTGAAAATTAGCTGCAA
AGGCTCCGGATATAGCTTCTCTTCTTACTGGATCCATTGGGTGCGCCAGATGCCGGGCAAAGGTCTCG
AGTGGATGGGCACTATCTTCCCGTCTCGTAGCTACACCACTTATAGCCCGAGCTTTCAGGGCCAGGTG
ACCATTAGCGCGGATAAAAGCATCAGCACCGCGTATCTGCAATGGAGCAGCCTGAAAGCGAGCGATAC
CGCGATGTATTATTGCGCGCGTGACACTCCGTCTACTGCTCTGAAATCTCCGTTCGATTACTGGGGCC
AAGGCACCCTGGTGACTGTTAGCTCAGCGTCGACCAAAGGCCCGAGCGTGTTTCCGCTGGCCCCGAGC
AGCAAAAGCACCAGCGGCGGCACCGCCGCACTGGGCTGCCTGGTGAAAGATTATTTCCCGGAACCAGT
GACCGTGAGCTGGAACAGCGGTGCCCTGACCAGCGGCGTGCATACCTTTCCGGCGGTGCTGCAAAGCA
GCGGCCTGTATAGCCTGAGCAGCGTTGTGACCGTGCCGAGCAGCAGCCTGGGCACCCAGACCTATATT
TGCAACGTCAACCATAAACCGAGCAACACCAAAGTCGATAAAAAAGTCGAACCGAAAAGCGAATTCAA
GGCTGAAATGCCTGTTCTGGAAAACCGGGCTGCTCAGGGCGATATTACTACACCCGGCGGTGCTCGCC
GTTTAACGGGTGATCAGACTGCCGCTCTGCGTGATTCTCTTAGCGATAAACCTGCAAAAAATATTATT
TTGCTGATTGGCGATGGGATGGGGGACTCGGAAATTACTGCCGCACGTAATTATGCCGAAGGTGCGGG
CGGCTTTTTTAAAGGTATAGATGCCTTACCGCTTACCGGGCAATACACTCACTATGCGCTGAATAGAA
AAACCGGCAAACCGGACTACGTCACCAGCTCGGCTGCATCAGCAACCGCCTGGTCAACCGGTGTCAAA
ACCTATAACGGCGCGCTGGGCGTCGATATTCACGAAAAAGATCACCCAACGATTCTGGAAATGGCAAA
AGCCGCAGGTCTGGCGACCGGTAACGTTTCTACCGCAGAGTTGCAGGATGCCACGCCCGCTGCGCTGG
TGGCACATGTGACCTCGCGCAAATGCTACGGTCCGAGCGCGACCAGTGAAAAATGTCCGGGTAACGCT
CTGGAAAAAGGCGGAAAAGGATCGATTACCGAACAGCTGCTTAACGCTCGTGCCGACGTTACGCTTGG
CGGCGGCGCAAAAACCTTTGCTGAAACGGCAACCGCTGGTGAATGGCAGGGAAAAACGCTGCGTGAAC
AGGCACAGGCGCGTGGTTATCAGTTGGTGAGCGATGCTGCCTCACTGAACTCGGTGACGGAAGCGAAT
CAGCAAAAACCCCTGCTTGGCCTGTTTGCTGACGGCAATATGCCAGTGCGCTGGCTAGGACCGAAAGC
AACGTACCATGGCAATATCGATAAGCCCGCAGTCACCTGTACGCCAAATCCGCAACGTAATGACAGTG
TACCAACCCTGGCGCAGATGACCGACAAAGCCATTGAATTGTTGAGTAAAAATGAGAAAGGCTTTTTC
CTGCAAGTTGAAGGTGCGTCAATCGATAAACAGGATCATGCTGCGAATCCTTGTGGGCAAATTGGCGA
GACGGTCGATCTCGATGAAGCCGTACAACGGGCGCTGGAGTTCGCTAAAAAGGAGGGTAACACGCTGG
TCATAGTCACCGCTGATCACGCCCACGCCAGCCAGATTGTTGCGCCGGATACCAAAGCTCCGGGCCTC
ACCCAGGCGCTAAATACCAAAGATGGCGCAGTGATGGTGATGAGTTACGGGAACTCCGAAGAGGATTC
ACAAGAACATACCGGCAGTCAGTTGCGTATTGCGGCGTATGGCCCGCATGCCGCCAATGTTGTTGGAC
TGACCGACCAGACCGATCTCTTCTACACCATGAAAGCCGCTCTGGGGCTGAAAGGCGCGCCGGACTAT
AAAGATGACGATGACAAAGGCGCGCCGCACCATCATCACCATCAC
SEQ ID NO: 20 shows the nucleic acid sequence encoding the light chain of the Fab region
of the anti-Ambra-1 antibody AbD33473;
GATATCGTGCTGACCCAGCCGCCGAGCGTGAGCGGTGCACCGGGCCAGCGCGTGACCATTAGCTGTAG
CGGCAGCAGCAGCAACATTGGTTACAACTACGTGTACTGGTACCAGCAGCTGCCGGGCACGGCGCCGA
AACTGCTGATCTACGAAAACAACAAACGCCCGAGCGGCGTGCCGGATCGCTTTAGCGGATCCAAAAGC
GGCACCAGCGCCAGCCTGGCGATTACCGGCCTGCAAGCAGAAGACGAAGCGGATTATTACTGCTCTTC
TTGGGACTCTCATTCTAACTCTTACGTGTTTGGCGGCGGCACGAAGTTAACCGTTCTTGGCCAGCCGA
AAGCCGCCCCAAGCGTGACCCTGTTTCCGCCGAGCAGCGAAGAACTGCAAGCCAACAAAGCCACCCTG
GTTTGCCTGATCAGCGATTTTTATCCGGGTGCCGTGACCGTGGCCTGGAAAGCCGATAGCAGCCCGGT
GAAAGCCGGCGTGGAAACCACCACCCCGAGCAAACAGAGCAACAACAAATATGCCGCCAGCAGCTATC
TGAGCCTGACCCCGGAACAGTGGAAAAGCCATCGCAGCTATAGTTGTCAAGTGACCCATGAAGGCAGC
ACCGTGGAAAAAACCGTGGCCCCGACCGAGGCC
SEQ ID NO: 21 shows the amino acid sequence of human Ambra-1;
MKVVPEKNAVRILWGRERGARAMGAQRLLQELVEDKTRWMKWEGKRVELPDSPRSTFLLAFSPDRILL
ASTHVNHNIYITEVKTGKCVHSLIGHRRTPWCVTFHPTISGLIASGCLDGEVRIWDLHGGSESWFTDS
NNAIASLAFHPTAQLLLIATANEIHFWDWSRREPFAVVKTASEMERVRLVRFDPLGHYLLTAIVNPSN
QQGDDEPEIPIDGTELSHYRQRALLQSQPVRRTPLLHNFLHMLSSRSSGIQVGEQSTVQDSATPSPPP
PPPQPSTERPRTSAYIRLRQRVSYPTAECCQHLGILCLCSRCSGTRVPSLLPHQDSVPPASARATTPS
FSFVQTEPFHPPEQASSTQQDQGLLNRPSAFSTVQSSTAGNTLRNLSLGPTRRSLGGPLSSHPSRYHR
EIAPGLTGSEWTRTVLSLNSRSEAESMPPPRTSASSVSLLSVLRQQEGGSQASVYTSATEGRGFPASG
LATESDGGNGSSQNNSGSIRHELQCDLRRFFLEYDRLQELDQSLSGEAPQTQQAQEMLNNNIESERPG
PSHQPTPHSSENNSNLSRGHLNRCRACHNLLTENNDTLRWERTTPNYSSGEASSSWQVPSSFESVPSS
GSQLPPLERTEGQTPSSSRLELSSSASPQEERTVGVAFNQETGHWERIYTQSSRSGTVSQEALHQDMP
EESSEEDSLRRRLLESSLISLSRYDGAGSREHPIYPDPARLSPAAYYAQRMIQYLSRRDSIRQRSMRY
QQNRLRSSTSSSSSDNQGPSVEGTDLEFEDFEDNGDRSRHRAPRNARMSAPSLGRFVPRRFLLPEYLP
YAGIFHERGQPGLATHSSVNRVLAGAVIGDGQSAVASNIANTTYRLQWWDFTKFDLPEISNASVNVLV
QNCKIYNDASCDISADGQLLAAFIPSSQRGFPDEGILAVYSLAPHNLGEMLYTKRFGPNAISVSLSPM
GRYVMVGLASRRILLHPSTEHMVAQVFRLQQAHGGETSMRRVFNVLYPMPADQRRHVSINSARWLPEP
GLGLAYGTNKGDLVICRPEALNSGVEYYWDQLNETVFTVHSNSRSSERPGTSRATWRTDRDMGLMNAI
GLQPRNPATSVTSQGTQTLALQLQNAETQTEREVPEPGTAASGPGEGEGSEYGASGEDALSRIQRLMA
EGGMTAVVQREQSTTMASMGGFGNNIIVSHRIHRSSQTGTEPGAAHTSSPQPSTSRGLLPEAGQLAER
GLSPRTASWDQPGTPGREPTQPTLPSSSPVPIPVSLPSAEGPTLHCELTNNNHLLDGGSSRGDAAGPR
GEPRNR
SEQ ID NO: 22 shows the amino acid sequence of the peptide to which anti-Ambra-1
antibodies were raised
CGGSSRGDAAGPRGEPRNR
SEQ ID NO: 23 shows the amino acid sequence of HCDR1 of anti-p62 antibody AbD34907;
TYYLH
SEQ ID NO: 24 shows the amino acid sequence of HCDR2 of the anti-p62 antibody
AbD34907;
LINPNNGGTNYAQKFQG
SEQ ID NO: 25 shows the amino acid sequence of HCDR3 of the anti-p62 antibody AbD34907;
GPGRLRPFGLYFDV
SEQ ID NO: 26 shows the amino acid sequence of LCDR1 of the anti-p62 antibody AbD34907;
RASQGIYTFLN
SEQ ID NO: 27 shows the amino acid sequence of LCDR2 of the anti-p62 antibody AbD34907;
AASTLOS
SEQ ID NO: 28 shows the amino acid sequence of LCDR3 of the anti-p62 antibody AbD34907;
FQYSSWPRT
SEQ ID NO: 29 shows the amino acid sequence of HCFR1 of the anti-p62 antibody AbD34907;;
QVOLVOSGAEVKKPGASVKVSCKASGYTFN
SEQ ID NO: 30 shows the amino acid sequence of HCFR2 of the anti-p62 antibody AbD34907;
WVRQAPGQGLEWMG
SEQ ID NO: 31 shows the amino acid sequence of HCFR3 of the anti-p62 antibody AbD34907;
RVTMTRDTSISTAYMELSRLRSEDTAVYYCAR
SEQ ID NO: 32 shows the amino acid sequence of HCFR4 of the anti-p62 antibody AbD34907;
WGQGTLVTVSS
SEQ ID NO: 33 shows the amino acid sequence of LCFR1 of the anti-p62 antibody AbD34907;
DIQMTQSPSSLSASVGDRVTITC
SEQ ID NO: 34 shows the amino acid sequence of LCFR2 of the anti-p62 antibody AbD34907;
WYQQKPGKAPKLLIY
SEQ ID NO: 35 shows the amino acid sequence of LCFR3 of the anti-p62 antibody AbD34907;
GVPSRFSGSGSGTDFTLTISSLOPEDFATYYC
SEQ ID NO: 36 shows the amino acid sequence of LCFR4 of the anti-p62 antibody AbD34907;
FGQGTKVEIK
SEQ ID NO: 37 shows the amino acid sequence of the VH domain of the anti-p62 antibody
AbD34907;
QVQLVQSGAEVKKPGASVKVSCKASGYTFNTYYLHWVRQAPGQGLEWMGLINPNNGGTNYAQKFQGRV
TMTRDTSISTAYMELSRLRSEDTAVYYCARGPGRLRPFGLYFDVWGQGTLVTVSS
SEQ ID NO: 38 shows the amino acid sequence of the VL domain of the anti-p62 antibody
AbD34907;
DIQMTQSPSSLSASVGDRVTITCRASQGIYTFLNWYQQKPGKAPKLLIYAASTLQSGVPSRFSGSGSG
TDFTLTISSLQPEDFATYYCFQYSSWPRTFGQGTKVEIK
SEQ ID NO: 39 shows the amino acid sequence of the Fd chain (VH domain and constant
domain) of the Fab region of the anti-p62 antibody AbD34907;
QVQLVQSGAEVKKPGASVKVSCKASGYTFNTYYLHWVRQAPGQGLEWMGLINPNNGGTNYAQKFQGRV
TMTRDTSISTAYMELSRLRSEDTAVYYCARGPGRLRPFGLYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTY
ICNVNHKPSNTKVDKKVEPKS
SEQ ID NO: 40 shows the amino acid sequence of the light chain (VL domain and constant
domain) of the Fab region of the anti-p62 antibody AbD34907;
DIQMTQSPSSLSASVGDRVTITCRASQGIYTFLNWYQQKPGKAPKLLIYAASTLQSGVPSRFSGSGSG
TDFTLTISSLQPEDFATYYCFQYSSWPRTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEA
SEQ ID NO: 41 shows the amino acid sequence of HCDR1 of anti-p62 antibody AbD34908;
NYWIG
SEQ ID NO: 42 shows the amino acid sequence of HCDR2 of the anti-p62 antibody
AbD34908;
IIDPSGSFTRYSPSFQG
SEQ ID NO: 43 shows the amino acid sequence of HCDR3 of the anti-p62 antibody AbD34908;
KYVPPVYSYWSYGTLDV
SEQ ID NO: 44 shows the amino acid sequence of LCDR1 of the anti-p62 antibody AbD34908;
RASQSVSASRLA
SEQ ID NO: 45 shows the amino acid sequence of LCDR2 of the anti-p62 antibody AbD34908;
GSSTRAT
SEQ ID NO: 46 shows the amino acid sequence of LCDR3 of the anti-p62 antibody AbD34908;
QQYLHLYT
SEQ ID NO: 47 shows the amino acid sequence of HCFR1 of the anti-p62 antibody AbD34908;
EVOLVQSGAEVKKPGESLKISCKGSGYSFT
SEQ ID NO: 48 shows the amino acid sequence of HCFR2 of the anti-p62 antibody AbD34908;
WVRQMPGKGLEWMG
SEQ ID NO: 49 shows the amino acid sequence of HCFR3 of the anti-p62 antibody AbD34908;
QVTISADKSISTAYLOWSSLKASDTAMYYCAR
SEQ ID NO: 50 shows the amino acid sequence of HCFR4 of the anti-p62 antibody AbD34908;
WGQGTLVTVSS
SEQ ID NO: 51 shows the amino acid sequence of LCFR1 of the anti-p62 antibody AbD34908;
DIVLTQSPATLSLSPGERATLSC
SEQ ID NO: 52 shows the amino acid sequence of LCFR2 of the anti-p62 antibody AbD34908;
WYQQKPGQAPRLLIY
SEQ ID NO: 53 shows the amino acid sequence of LCFR3 of the anti-p62 antibody AbD34908;
GIPARFSGSGSGTDFTLTISSLEPEDFAVYYC
SEQ ID NO: 54 shows the amino acid sequence of LCFR4 of the anti-p62 antibody AbD34908;
FGQGTKVEIK
SEQ ID NO: 55 shows the amino acid sequence of the VH domain of the anti-p62 antibody
AbD34908;
EVQLVQSGAEVKKPGESLKISCKGSGYSFTNYWIGWVRQMPGKGLEWMGIIDPSGSFTRYSPSFQGQV
TISADKSISTAYLQWSSLKASDTAMYYCARKYVPPVYSYWSYGTLDVWGQGTLVTVSS
SEQ ID NO: 56 shows the amino acid sequence of the VL domain of the anti-p62 antibody
AbD34908;
DIVLTQSPATLSLSPGERATLSCRASQSVSASRLAWYQQKPGQAPRLLIYGSSTRATGIPARFSGSGS
GTDFTLTISSLEPEDFAVYYCQQYLHLYTFGQGTKVEIK
SEQ ID NO: 57 shows the amino acid sequence of the Fd chain (VH domain and constant
domain) of the Fab region of the anti-p62 antibody AbD34908;
EVQLVQSGAEVKKPGESLKISCKGSGYSFTNYWIGWVRQMPGKGLEWMGIIDPSGSFTRYSPSFQGQV
TISADKSISTAYLQWSSLKASDTAMYYCARKYVPPVYSYWSYGTLDVWGQGTLVTVSSASTKGPSVFP
LAPSSKSTSGGTAALGCLVKDYFPEPVTVSWNSGALTSGVHTFPAVLQSSGLYSLSSVVTVPSSSLGT
QTYICNVNHKPSNTKVDKKVEPKS
SEQ ID NO: 58 shows the amino acid sequence of the light chain (VL domain and constant
domain) of the Fab region of the anti-p62 antibody AbD34908;
DIVLTQSPATLSLSPGERATLSCRASQSVSASRLAWYQQKPGQAPRLLIYGSSTRATGIPARFSGSGS
GTDFTLTISSLEPEDFAVYYCQQYLHLYTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEA
SEQ ID NO: 59 shows full amino acid sequence of the anti-p62 antibody AbD34907
including alkaline phosphatase dimerization domain sequence (AP), FLAG and His6 tag;
DIQMTQSPSSLSASVGDRVTITCRASQGIYTFLNWYQQKPGKAPKLLIYAASTLQSGVPSRFSGSGSG
TDFTLTISSLQPEDFATYYCFQYSSWPRTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEA*QVQLVQSGAEVKKPGASVKVSCKASGYTFNTYYLHWVRQAPGQGLEWMGLINPNNGG
TNYAQKFQGRVTMTRDTSISTAYMELSRLRSEDTAVYYCARGPGRLRPFGLYFDVWGQGTLVTVSSAS
TKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSEFKAEMPVLENRAAQGDITTPGGARRLTGDQTAALR
DSLSDKPAKNIILLIGDGMGDSEITAARNYAEGAGGFFKGIDALPLTGQYTHYALNRKTGKPDYVTSS
AASATAWSTGVKTYNGALGVDIHEKDHPTILEMAKAAGLATGNVSTAELQDATPAALVAHVTSRKCYG
PSATSEKCPGNALEKGGKGSITEQLLNARADVTLGGGAKTFAETATAGEWQGKTLREQAQARGYQLVS
DAASLNSVTEANQQKPLLGLFADGNMPVRWLGPKATYHGNIDKPAVTCTPNPQRNDSVPTLAQMTDKA
IELLSKNEKGFFLQVEGASIDKQDHAANPCGQIGETVDLDEAVQRALEFAKKEGNTLVIVTADHAHAS
QIVAPDTKAPGLTQALNTKDGAVMVMSYGNSEEDSQEHTGSQLRIAAYGPHAANVVGLTDQTDLFYTM
KAALGLKGAPDYKDDDDKGAPHHHHHH
SEQ ID NO: 60 shows full amino acid sequence of the anti-p62 antibody AbD34908
including alkaline phosphatase dimerization domain sequence (AP), FLAG and His6 tag;
DIVLTQSPATLSLSPGERATLSCRASQSVSASRLAWYQQKPGQAPRLLIYGSSTRATGIPARFSGSGS
GTDFTLTISSLEPEDFAVYYCQQYLHLYTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEA*EVQLVQSGAEVKKPGESLKISCKGSGYSFTNYWIGWVRQMPGKGLEWMGIIDPSGSF
TRYSPSFQGQVTISADKSISTAYLQWSSLKASDTAMYYCARKYVPPVYSYWSYGTLDVWGQGTLVTVS
SASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSEFKAEMPVLENRAAQGDITTPGGARRLTGDQTA
ALRDSLSDKPAKNIILLIGDGMGDSEITAARNYAEGAGGFFKGIDALPLTGQYTHYALNRKTGKPDYV
TSSAASATAWSTGVKTYNGALGVDIHEKDHPTILEMAKAAGLATGNVSTAELQDATPAALVAHVTSRK
CYGPSATSEKCPGNALEKGGKGSITEQLLNARADVTLGGGAKTFAETATAGEWQGKTLREQAQARGYQ
LVSDAASLNSVTEANQQKPLLGLFADGNMPVRWLGPKATYHGNIDKPAVTCTPNPQRNDSVPTLAQMT
DKAIELLSKNEKGFFLQVEGASIDKQDHAANPCGQIGETVDLDEAVQRALEFAKKEGNTLVIVTADHA
HASQIVAPDTKAPGLTQALNTKDGAVMVMSYGNSEEDSQEHTGSQLRIAAYGPHAANVVGLTDQTDLF
YTMKAALGLKGAPDYKDDDDKGAPHHHHHH
SEQ ID NO: 61 shows the amino acid sequence of human p62;
MASLTVKAYLLGKEDAAREIRRFSFCCSPEPEAEAEAAAGPGPCERLLSRVAALFPALRPGGFQAHYR
DEDGDLVAFSSDEELTMAMSYVKDDIFRIYIKEKKECRRDHRPPCAQEAPRNMVHPNVICDGCNGPVV
GTRYKCSVCPDYDLCSVCEGKGLHRGHTKLAFPSPFGHLSEGFSHSRWLRKVKHGHFGWPGWEMGPPG
NWSPRPPRAGEARPGPTAESASGPSEDPSVNFLKNVGESVAAALSPLGIEVDIDVEHGGKRSRLTPVS
PESSSTEEKSSSQPSSCCSDPSKPGGNVEGATQSLAEQMRKIALESEGRPEEQMESDNCSGGDDDWTH
LSSKEVDPSTGELQSLQMPESEGPSSLDPSQEGPTGLKEAALYPHLPPEADPRLIESLSQMLSMGFSD
EGGWLTRLLQTKNYDIGAALDTIQYSKHPPPL
SEQ ID NO: 62 shows the p62 protein fragment used to generate anti-p62 antibodies;
MAMSYVKDDIFRIYIKEKKECRRDHRPPCAQEAPRNMVHPNVICDGCNGPVVGTRYKCSVCPDYDLCS
VCEGKGLHRGHTKLAFPSPFGHLSEGFSHSRWLRKVKHGHFGWPGWEMGPPGNWSPRPPRAGEARPGP
TAESASGPSEDPSVNFLKNVGESVAAALSPLGIEVDIDVEHGGKRSRLTPVSPESSSTEEKSSSQPSS
CCSDPSKPGGNVEGATQSLAEQMRKIALESEGRPEEQMESDNCSGGDDDWTHLSSKEVDPSTGELQSL
QMPESEGPSSLDPSQEGPTGLKEAALYPHLPPEADPRLIESLSQMLSMGFSDEGGWLTRLLQTKNYDI
GAALDTIQYSKHPPPLLEHHHHHH
SEQ ID NO: 63 shows the Ambra-1 C-terminal sequence used for replacement analysis in
epitope mapping:
VSLPSAEGPTLHCELTNNNHLLDGGSSRGDAAGPRGEPRNR
SEQ ID NO: 64 shows the REPNET epitope of the Anti-Ambra-1 antibodies:
DGGSSRGDAAGPRGEPRNR
SEQ ID NO: 65 shows the LIN epitope of the Anti-Ambra-1 antibodies:
HLLDGGSSR
SEQ ID NO: 66 shows the LOOP epitope of the Anti-Ambra-1 antibodies:
NHLLDGGSSR
SEQ ID NO: 67 shows a core epitope of the Anti-Ambra-1 antibodies:
EPRN
SEQ ID NO: 68 shows a core epitope of the Anti-Ambra-1 antibodies:
EPR
SEQ ID NO: 69 show the Flag epitope tag:
DYKDDDDK
SEQ ID NO: 70 show the V5 epitope tag:
GKP IPNPLLGLDST
SEQ ID NO: 71 show the Strep epitope tag:
WSHPQFEK

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al,. 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2nd Edition edited by Mark Gibson) and Pharmaceutical Excipients: Properties, Functionality and Applications in Research and Industry (edited by Otilia M Y Koo, published by Wiley). Most general pharmacological techniques can be found in A Textbook of Clinical Pharmacology and Therapeutics (5th Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure. For chemical terms, the skilled person may refer to the International Union of Pure and Applied Chemistry (IUPAC). Units, prefixes and symbols are denoted in their Système International d′Unités (SI) accepted form. Numeric ranges are inclusive of the numbers defining the range.

Methods for prognostic classification of SCC

In certain embodiments, the invention provides a method for determining whether a subject with SCC has an increased risk of progression and/or recurrence of SCC.

Typically, the method is in vitro. The subject may be a human or an animal suffering from SCC. In some embodiments, the subject is a horse, cat or dog. Typically, the subject is a human. In some embodiments, the subject has already been diagnosed as having SCC.

As used herein, the term “SCC” (i.e., epidermoid carcinoma) refers to a form of cancer arising from squamous cells. Squamous cells may be found in the tissue that forms the surface of the skin, the lining of hollow organs in the body and the lining of the respiratory and digestive tracts.

Certain embodiments of the invention may provide methods of determining whether a subject has an increased risk of progression and/or recurrence of any type of SCC. In certain embodiments, the SCC is cutaneous SCC. As used herein, the term “cutaneous squamous cell carcinoma (cSCC)” refers to a form of skin cancer arising from malignant proliferation of the keratinocyte cells of the epidermis, which may mediate cellular invasion. Disease-related cell invasion and/or proliferation may be any abnormal, undesirable or pathological cell invasion and/or proliferation, for example tumour-related cell invasion and/or proliferation.

Treatment for early-stage SCC typically involves surgery to excise the tumour(s). Therapy is generally used to control the spread of metastases in the later stages of the disease, by which time the prognosis is typically poor. The identification of those subjects who are in the early stages of the disease but who are at a high or increased risk of tumour progression and/or recurrence would advantageously enable treatment to be tailored accordingly. For example, therapy may then be administered to those subjects sooner than it might normally be administered, thereby inhibiting, preventing or delaying tumour progression and/or recurrence and improving the prognosis of those subjects.

Thus, in some embodiments, the subject, prior to identification, is ineligible for therapeutic agent treatment. In certain embodiments, a subject can be put forward for a treatment regime at an earlier or less progressed stage as compared to the prior art methods of treating carcinoma in which a patient is only treated with a therapeutic agent when they are suffering at a later stage.

As used herein, “an increased risk of progression” of SCC refers to the risk of the SCC progressing from the epidermis to areas below the skin such as lymph nodes, muscle, bone, cartilage and/or metastasizing to distant sites.

As used herein, the term “recurrence” of SCC refers to the risk of the SCC returning, for example, after excision of the tumor by surgery.

In certain embodiments, the methods described herein relate to determining whether a subject has an increased risk of metastasis. the term “metastasis” refers to the recurrence or disease progression that may occur locally (such as local recurrence and in transit disease), regionally (such as nodal micro-metastasis or macro-metastasis), or distally (such as brain, lung and other tissues). In some embodiments, the term “metastasis” is used to refer to metastatic disease following a primary carcinoma. Typically, metastasis originating from a primary carcinoma may spread to the lungs and/or brain of the subject as well as other locations.

As used herein, the term “primary tumour” or “primary carcinoma” refers to a malignant tumour on the skin at the site of origin, regardless of thickness, in patients without clinical or histologic evidence of regional or distant metastatic disease.

As used herein, the term “increased risk of metastasis” refers to an increased chance of SCC spreading from the primary site.

The stage of SCC is a description of how widespread it is. This includes its thickness (e.g. in the skin or other organ), whether it has spread to nearby lymph nodes or any other organs, and certain other factors. The stage is based on the results of the physical exam, biopsies, and any imaging tests (CT or MRI scan, etc.) or other tests that have been done. Such tests will be known to those skilled in the art. For example, the American Joint Commission on Cancer (AJCC) TNM may be used to stage cSCC (J Am Acad Dermatol. 2011 64 (6) 1051-1059). Table 1 below describes the features identifying each stage.

TABLE 1
AJCC 7^{th} edition cSCC tumor staging system¹.

| Stage | |
|---|---|
| TX | Primary tumor cannot be assessed |
| T0 | No evidence of primary tumor |
| Tis | Carcinoma in situ |
| T1 | Tumor ≤ 2 cm in greatest dimension with <2 high-risk features² |
| T2 | Tumor > 2 cm in greatest dimension with or without one |
| | additional high-risk feature, or any size with >2 high-risk |
| | features² |
| T3 | Tumor with invasion of maxilla, mandible, orbit, or temporal |
| | bone |
| T4 | Tumor with invasion of skeleton (axial or appendicular) or |
| | perineural invasion of skull base. |

¹In the absence of nodal or distant metastasis, Tis = Stage 0, T1 = Stage I, and T2 = Stage II.
²High-risk features include depth > 2 mm or Clark level ≥ 4, perineural invasion, location on the ear or lip, and poor differentiation.

In some embodiments, the subject is suffering from TX, TO, Tis, T1 (i.e. Stage I) or T2 (i.e. Stage II) SCC. In some embodiments, the methods further comprise staging a primary carcinoma present in the tissue sample obtained from the subject in accordance with AJCC staging. Typically, a subject is selected who is suffering from AJCC T1 or T2 SCC.

In some embodiments, an alternative SCC staging system is used. For example, the staging system used by Breuninger et al. (J Dtsch Dermatol Ges. 2012 10 (8) 579-586), the Brigham and Woman's Hospital (BWH) staging system (J Clin Oncol. 2014 32 (4) 327-334) or the AJCC 8th edition (AJCC 8) staging system (CA Cancer J Clin. 2017 67 (2): 122-137) may alternatively be used to stage a primary tissue present in the tissue sample.

In the Breuninger system, the tumour may be staged primarily based on clinical tumour size and histological tumour thickness. Typically, a subject is selected who has a tumour >2 cm in diameter and thickness ≥6 mm.

In the BHW system, the tumour may be staged primarily based on four risk factors (namely tumour diameter, tumour diameter ≥2 cm, invasion beyond subcutaneous fat, poorly differentiated and perineural invasion. Typically, a subject is selected who is T1 (no high-risk factors), T2a (1 high-risk factor), T2b (2-3 high risk factors) or T3 (4 high risk factors).

In the AJCC8 system (head and neck only), typically a subject is selected who is T1 (tumour diameter <2 cm), T2 (tumour diameter ≥2 cm and <4 cm in greatest dimension, T3 (tumour diameter ≥4 cm, or minor bone erosion, or perineural invasion, or deep invasion) or T4 (tumour with gross cortical bone/marrow invasion.

### Tissue Analysis

In certain embodiments, the method for determining whether a subject has an increased risk of progression and/or recurrence of SCC comprises determining the expression of Ambra-1 and p62 in one or more tissue samples obtained from the subject. Typically, the tissue sample(s) comprise at least one primary tumour and/or tissue overlying the primary tumour. For example, the tissue sample may comprise at least a portion of SCC tissue and/or cells and expression of Ambra-1 and p62 is determined in the tissue and/or cells.

In certain embodiments, the tissue sample(s) may also comprise non-carcinoma tissue, e.g. normal tissue adjacent the primary carcinoma.

In some embodiments, the tissue sample has previously been obtained from the subject such that the sampling itself does not form a part of the methods of the invention. The sample may have been obtained immediately prior to the method, or hours, days or weeks prior to the method. In other embodiments, a method of the invention may additionally comprise the step of obtaining the tissue sample from the subject.

Typically, the tissue sample may be a biopsy, or a section thereof, obtained from the subject. A tissue sample, such as a biopsy, can be obtained through a variety of sampling methods known to those skilled in the art, including a punch biopsy, shave biopsy, wide local excision and other means. Aptly, the tumour sample is taken from a surgical site from which the primary carcinoma has been excised from the subject.

Typically, the tissue sample may be frozen, fresh, fixed (e.g. formalin fixed), centrifuged, and/or embedded (e.g. paraffin embedded), etc. The tissue sample may be or have been subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of Ambra-1 and/or p62 in the sample. Likewise, biopsies may also be subjected to post-collection preparative and storage techniques, e.g., fixation. A tissue sample, or a section thereof, may be mounted on a solid support, such as a slide.

In certain embodiments, the expression of Ambra-1 and p62 are determined in the same tissue sample(s) obtained from the subject. Alternatively, the expression of Ambra-1 and p62 may be determined in different tissue sample(s) obtained from the subject. For example, Ambra-1 expression may be determined in a first sample obtained from the subject, and p62 expression may be determined in a second sample obtained from the subject.

In certain embodiments, the expression of Ambra-1 and p62 are determined at the same time. Alternatively, the expression of Ambra-1 and p62 may be determined at different times.

In certain embodiments, the expression of Ambra-1 and/or p62 is determined in the nucleus of cells within the tissue. Preferably, expression of Ambra-1 and/or p62 is determined in the cytoplasm of cells within the tissue. For example, expression of both Ambra-1 and p62 may be determined in cytoplasm of cells within the tissue.

The expression of Ambra-1 and/or p62 may be determined in any one or more regions of the tissue sample. In certain embodiments, the expression of Ambra-1 and/or p62 may be determined in the normal epidermis. As used herein, the term “normal epidermis” is understood to mean the epidermal region distant to the primary tumour.

In certain embodiments, the expression of Ambra-1 and/or p62 may be determined in the peritumoral epidermis. As used herein, the term “peritumoral epidermis” is understood to mean the epidermal region directly alongside the primary tumour.

In certain embodiments, the expression of Ambra-1 and/or p62 may be determined in the tumour mass. As used herein, the term “tumour mass” is understood to mean the principle tumour area but not the deepest aspect of the tumour or displaying an invasive phenotype.

In certain embodiments, the expression of Ambra-1 and/or p62 may be determined in the tumour growth front. As used herein, the term “tumour growth front” is understood to mean the tumour areas at the deepest aspect of the tumour or displaying an invasive phenotype. In certain embodiments, the expression of Ambra-1 and/or p62 is determined in each of the normal epidermis, peritumoral epidermis, tumour mass and/or tumour growth front of the tissue.

In preferred embodiments, the expression of Ambra-1 (e.g., in the cytoplasm) is determined in the tumour mass and/or growth front. Most preferably, the expression of Ambra-1 (e.g., in the cytoplasm) is determined in the tumour growth front.

In preferred embodiments, the expression of p62 (e.g., in the cytoplasm) is determined in the cells of the normal and/or peritumoral epidermis. Most preferably, the expression of p62 (e.g., in the cytoplasm) is determined in the peritumoral epidermis.

In the most preferred embodiments, expression of cytoplasmic p62 is determined in the peritumoral epidermis and expression of cytoplasmic Ambra-1 is determined in the tumour growth front.

According to any method described herein, the differentiation status of the primary tumour may be determined before or after determining the expression of Ambra-1 and p62 in the one or more tissue samples. For example, the primary tumour may be categorised as poorly, moderately or well differentiated depending on the resemblance of the tissue. Any suitable technique may be used to objectively grade the differentiation of SCC, including those described, for example, by Lindelof et al., 2006 (Acta Derm Venereol. 86 (3) 219-22).

As used herein, the term “poorly-differentiated” may refer to tumours wherein about 25% or less of cells are differentiated.

As used herein, the term “moderately differentiated” may refer to tumours wherein between about 25% to about 75% of cells are differentiated.

As used herein, the term “well-differentiated” may refer to tumours wherein more than about 75% of cells are differentiated.

In preferred embodiments, the primary tumour is categorised as “moderately or poorly differentiated” before or after determining the expression of Ambra-1 and p62. For example, such tumours may comprise about 75% or less differentiated cells.

In certain embodiments, the invention comprises a method of determining whether a subject has an increased risk of progression and/or recurrence of SCC, the method comprising:
(i) determining the differentiation status of a primary tumour within one or more tissue samples obtained from the subject, and
(ii) determining the expression of Ambra-1 and p62 in one or more tissue samples obtained from the subject, wherein the tissue sample(s) comprises the primary tumour and tissue overlying the primary tumour, wherein (a) a moderately or poorly differentiated primary tumour and (b) a decrease or loss in the expression of Ambra-1 and a decrease or loss in the expression of p62 in the tissue sample(s) compared to one or more reference level(s) is indicative of an increased risk of progression and/or recurrence of the tumour.

In such embodiments, expression of Ambra-1 and/or p62 is typically determined in the cytoplasm of cells within the SCC tissue biopsy as further described herein.

In such embodiments, expression of Ambra-1 (e.g., cytoplasmic Ambra-1) is determined in the primary tumour as further described herein. Preferably, expression of Ambra-1 (e.g., cytoplasmic Ambra-1) is determined in the tumour growth front of the primary tumour as further described herein.

In such embodiments, expression of p62 (e.g., cytoplasmic p62) is typically determined in the tissue overlying the primary tumour as further described herein. Preferably, expression of p62 (e.g., cytoplasmic p62) is determined in the peritumoral epidermis overlying the primary tumour as further described herein.

In such embodiments, the subject typically has cutaneous SCC (cSCC).

### Determining Ambra-1 and p62 Expression

Ambra-1 (activating molecule in Beclin-1 regulated autophagy protein 1) is a WD40-containing protein. Studies have implicated Ambra-1 in the control of autophagy and cellular differentiation. The full length human amino acid sequence of Ambra-1 is set forth in SEQ ID NO: 21.

p62 (also known as sequestosome-1/SQSTM1) is a multidomain adaptor protein transporting ubiquitinated proteins during autophagy. The full length human amino acid sequence of p62 is set forth in SEQ ID NO: 61.

Unexpectedly, the inventors have identified in a discovery cohort a novel correlation between the expression levels of Ambra-1 and p62 and the likelihood of progression and/or recurrence of SCC in subjects. In particular, the inventors have shown that a decrease or loss of expression of Ambra-1 and decrease or loss of expression of p62 in tissue comprising at least a portion of the primary tumour indicates that the subject has an increased risk of progression and/or recurrence of the disease.

In certain embodiments, determining the expression of Ambra-1 and p62 in the tissue sample comprises:
contacting the tissue with a first ligand specific for Ambra-1, wherein the presence of Ambra-1 creates an Ambra-1-ligand complex;
contacting the tissue with a second ligand specific for p62, wherein the presence of p62 creates a p62-ligand complex; and
detecting and/or quantifying the Ambra-1-ligand complex and the p62-ligand complex.

In some embodiments, the first ligand comprises an anti-Ambra-1 antibody or aptamer. In some embodiments, the anti-Ambra-1 antibody or aptamer binds specifically to a protein (or region thereof) having the sequence shown in SEQ ID NO: 21, 22, 63, 64, 65, 66, 67 and/or 68.

As used herein, the term “aptamer” refers to a non-naturally occurring oligonucleotide that can form a three-dimensional structure that binds to another molecule with high affinity and specificity. As used herein, the terms “nucleic acid ligand” and “aptamer” may be used interchangeably. Aptamer specificity may be comparable or even higher than that of an antibody.

Aptamers may fold into a variety of complex, sequence-specific tertiary conformations, meaning that they can bind a wide range of targets and rival antibodies in their potential diversity. It is recognized that affinity interactions are a matter of degree; however, in this context, the “specific binding affinity” of an aptamer for its target means that the aptamer binds to its target, i.e. a target molecule, generally with a much higher degree of affinity than it binds to other, non-target, components in a mixture or sample.

An “aptamer” or “nucleic acid ligand” is a set of copies of one type or species of nucleic acid molecule that has a particular nucleotide sequence. An aptamer can include any suitable number of nucleotides. “Aptamers” refers to more than one such set of molecules. Different aptamers can have either the same or different numbers of nucleotides. Aptamers may be DNA or RNA and may be single stranded, double stranded, or contain double stranded regions. In one embodiment, the aptamer comprises single-stranded DNA (ss-DNA) or single stranded RNA (ss-RNA).

In some embodiments, the second ligand comprises an anti-p62 antibody or aptamer. In some embodiments, the anti-p62 antibody or aptamer binds specifically to a protein having the sequence shown in SEQ ID NO: 61 or 62.

In certain embodiments, the anti-Ambra-1 or p62 antibody is an antibody as further described herein.

The amino acid sequences of human Ambra-1 and p62 are provided herein as examples. However, it will be appreciated that variants of these sequences may be known or identified.

In some embodiments, the subject is a non-human mammal. It should therefore also be appreciated that references herein to Ambra-1 and p62 include the sequences of non-human homologues thereof.

In some embodiments, the first ligand comprises a first detection moiety and the second ligand comprises a second detection moiety. The first detection moiety may be the same as the second detection moiety, or it may be different.

In some embodiments, the method comprises contacting a first portion or section of the tissue sample with the first ligand and contacting a second portion or section of the tissue sample with the second ligand. This is particularly suitable for embodiments wherein the first detection moiety is the same as the second detection moiety. In some alternative embodiments, the method comprises contacting the tissue sample, or a portion or section thereof, with the first ligand and contacting the same tissue sample, or portion or section thereof, with the second ligand. It may be possible to detect and/or quantify both the Ambra-1-ligand complex and the p62-ligand complex in the same sample, or portion or section thereof, particularly if the first and second detection moieties are different.

Aptly, the first and/or second ligands may be used in combination with one or more capture agents. Thus, in some embodiments, the step of detecting and/or quantifying the Ambra-1-ligand complex and the p62-ligand complex comprises contacting the tissue sample(s) (or the section(s) or portion(s) thereof) with at least one capture agent. Aptly, a first capture agent which binds specifically to the first ligand may be used to detect and/or quantify the Ambra-1-ligand complex, while a second capture agent which binds specifically to the second ligand may be used to the detect and/or quantify the p62-ligand complex. Alternatively, a single capture agent may be used which is capable of binding specifically to both the first and second ligands.

In some embodiments, the capture agent comprises a binding moiety and a detection moiety. In some embodiments, the binding moiety is a secondary antibody which binds specifically to the first and/or second ligands. For example, the binding moiety may be a universal anti-IgG antibody that is capable of binding to primary antibodies used as the first and second ligands.

In some embodiments, the method further comprises one or more wash steps to remove unbound first and second ligands and, optionally, unbound capture agents.

In certain embodiments, the expression of Ambra-1 and/or p62 is determined using an antibody (or aptamer) against Ambra-1 and/or an antibody (or aptamer) against p62 as further described herein. Typically, the expression of Ambra-1 and/or p62 is determined by contacting the tissue with the antibodies (or aptamers) and detecting the presence of the bound antibodies (or aptamers). For example, presence of the bound antibodies (or aptamers) may be detected by visualising the antibodies (or aptamers) in the tissue sample with reagent(s) that generate detectable signal(s) (e.g. detection moieties as described herein).

In some embodiments, the method comprises contacting the tissue with the antibody (or aptamer) under conditions permissive for binding of the anti-Ambra-1 antibody (or aptamer) and/or anti-p62 antibody (or aptamer) to Ambra-1 and/or p62 and detecting whether a complex is formed between the anti-Ambra-1 antibody (or aptamer) and/or anti-p62 antibody (or aptamer) and Ambra-1 and/or p62 respectively.

In some embodiments, the first and/or second ligand comprises a detection moiety (e.g. a fluorescent label). A detection moiety enables the direct or indirect detection and/or quantification of the complexes formed.

In certain embodiments, determining the expression of Ambra-1 and p62 in the tissue sample comprises measuring the levels of each of the proteins present in the tissue. This may be achieved by methods known to those skilled in the art. Such methods include immunoassays, for example immunohistochemistry (IHC), immunofluorescence (IF), immunoblotting, flow cytometry (e.g., FACS™) or enzyme-linked immunosorbent assay (ELISA), and the like.

In certain embodiments, the expression of Ambra-1 and/or p62 is determined by automated immunohistochemistry. For example, robotically automated immunohistochemistry apparatus may be used. Such apparatus may automatically section the tissue, prepare slides, perform immunohistochemistry procedure and detect intensity of immunostaining (e.g., the intensity of the Ambra-1 and/or p62 antibodies or aptamers binding to Ambra-1 and/or p62 present in the tissue). Typically, such apparatus may also output data, e.g., output an indication of the expression level(s) and this indicates whether the subject suffering from SCC has an increased risk of progression and/or recurrence of a tumour.

Automated immunohistochemistry apparatus are commercially available and include, for example, Autostainers 360, 480, 720 and Labvision PT module machines from LabVision Corporation. Other apparatus include, for example, BOND™ Automated IHC & In Situ Hybridization System, Automate slide loader from GTI vision.

Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI).

Automated immunohistochemistry systems such as NexES® automated IHC slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus).

In the method of determining whether a subject has an increased risk of progression and/or recurrence of SCC, a decrease or loss in expression of Ambra-1 and a decrease or loss in expression of p62 in the tissue sample is indicative of an increased risk of progression and/or recurrence of the tumour (e.g. a poor prognosis for the subject).

For example, a decrease or loss in the expression of Ambra-1 may be a level of expression of Ambra-1 less than about 75%, less than about 50%, less than about 25%, less than about 10%, less than about 5% or less of one or more reference level(s). Typically, a decrease in expression of Ambra-1 is a level of expression of Ambra-1 from about 25% to about 75% compared to the reference level. Typically, a loss of expression of Ambra-1 is a level of expression of Ambra-1 less than about 25% compared to the reference level.

In some embodiments, there is substantially no expression of Ambra-1 in the tissue sample.

The expression of Ambra-1 may be determined in the nucleus and/or cytoplasm of cells within the tissue as further described herein. For example, expression of Ambra-1 may be determined in the normal epidermis, peritumoral epidermis, tumour mass and/or tumour growth front of the tissue. Typically, the expression of Ambra-1 is determined in the tumour mass and/or tumour growth front of the tissue. Preferably, cytoplasmic Ambra-1 is determined in the tumour growth front of the tissue.

In certain embodiments, an increase in the expression of Ambra-1 in the tissue sample compared to the reference tissue or levels (e.g. normal tissue) is indicative of a low risk of progression and/or recurrence of a tumour.

For example, an increase in the expression of Ambra-1 in the tissue sample compared to a reference level may be a level of expression of Ambra-1 from about 125%, 150%, 175%, 200%, 300%, 400%, 500% or more of the reference level.

In certain embodiments, no significant difference in the expression of Ambra-1 in the tissue sample compared to a reference level is indicative of a low risk of progression and/or recurrence of a tumour. For example, no significant difference in the expression of Ambra-1 in the tissue sample compared to the reference levels may be a level of Ambra-1 greater than about 75% of the reference level. In particular, the expression level of Ambra-1 in the tissue sample may be from about 75% to 125% as compared to the reference levels.

Similarly, a decrease or loss in the expression of p62 may be a level of expression of p62 less than about 75%, less than about 50%, less than about 25%, less than about 10%, less than about 5% or less of one or more reference level(s). Typically, a decrease in expression of p62 is a level of expression of p62 from about 25% to about 75% compared to the reference level. Typically, a loss of expression of p62 is a level of expression of p62 less than about 25% compared to the reference level.

In some embodiments, there is substantially no expression of p62 in the tissue sample.

The expression of p62 may be determined in the nucleus and/or cytoplasm of cells within the tissue as further described herein. For example, expression of p62 may be determined in the normal epidermis, peritumoral epidermis, tumour mass and/or tumour growth front of the tissue. Typically, the expression of p62 is determined in the normal epidermis and/or peritumoral epidermis of the tissue. Preferably, cytoplasmic p62 is determined in the peritumoral epidermis of the tissue.

In certain embodiments, an increase in the expression of p62 in the tissue sample compared to the reference levels is indicative of a low risk of progression and/or recurrence of a tumour.

For example, an increase in the expression of p62 in the tissue sample compared to a reference level may be a level of expression of p62 from about 125%, 150%, 175%, 200%, 300%, 400%, 500% or more of the reference level.

In certain embodiments, no significant difference in the expression of p62 in the tissue sample compared to a reference level is indicative of a low risk of progression and/or recurrence of a tumour. For example, no significant difference in the expression of p62 in the tissue sample compared to the reference levels may be a level of p62 greater than about 75% of the reference levels. In particular, the expression level of p62 in the tissue sample may be from about 75% to 125% as compared to the reference levels.

The term “comparing” and “compare” may refer to a comparison of corresponding parameters or levels, e.g., an absolute amount is compared to an absolute reference amount while a concentration is compared to a reference concentration or a signal intensity signal obtained from the tissue sample is compared to the same type of signal intensity obtained from the reference. The comparison may be carried out manually, for example by visual assessment, or it may be automated (e.g. using an automated scanner or computer-assisted). Thus, the comparison may be carried out by a computing device.

In certain embodiments, the expression of Ambra-1 and/or p62 is scored on the basis of the intensity and/or proportion of positive cells in the tissue sample (or defined region of the tissue sample as further described herein). Scoring methods have been described and are well known to one of ordinary skill in the art.

In one embodiment, an intensity score may be defined as follows: 0=no appreciable staining in the cells, 1=faint/barely perceptible partial staining in the cytoplasm and/or nucleus of the cells, 2=weak to moderate staining of the cytoplasm and/or nucleus of the cells, and 3=strong staining of the cytoplasm and/or nucleus of the cells. A proportion score may be defined as follows: 0=less than 5%, 1=from 5% to 25%, 2=from 26% to 50%, 3=from 51% to 75%, and 4=more than 75%. A total score may be calculated by multiplying the intensity score and the proportion score, producing a total range of 0 to 12. In certain embodiments, scores of 0 to 3 may be indicative of decrease or loss of expression. In certain embodiments, scores of 4 to 12 may be indicative of an increase in expression.

In another embodiment, a H-score may be calculated (McCarty et al., Cancer Res. 1986 46 (8 Supl): 4244s-4248s). An intensity score may be defined as follows:

| | |
|---|---|
| 1= | faint/barely perceptible partial (e.g. weak) staining in the cells; |
| 2= | moderate staining of the cells; |
| 3= | strong staining of the cells. |

The H score combines components of staining intensity with the percentage of positive cells. It has a value between 0 and 300 and is defined as:
1*(percentage of cells staining at 1+intensity);
+2*(percentage of cells staining at 2+intensity);
+3*(percentage of cells staining at 3+intensity);
=H score.

Generally, a H-score of about 100 or above may indicate increased expression of Ambra-1 and/or p62. Generally, a H-score of less than about 100 may indicate decreased expression of Ambra-1 and/or p62. However, more specific cut-off values may be determined for any given type of tissue as further described herein.

In certain embodiments, cytoplasmic expression of Ambra-1 and/or p62 is quantified using a cytoplasmic algorithm (e.g., a cytoplasmic V2 algorithm). Such an algorithm may detect the cytoplasmic staining for the individual cells in the cytoplasm and quantify their intensity.

For example, a cytoplasmic intensity score (e.g., H-score) derived from the average intensity of the staining of the cytoplasm (cellular average) may be determined according to threshold intervals set in the algorithm macro. In one embodiment, cytoplasmic staining may be classified as 0, 1+, 2+ and 3+based on cytoplasmic staining intensity. A cell may be classified 0 when it has no cytoplasmic staining. A cell may be classified 1+when it has weak cytoplasmic staining. A cell may be classified 2+when it has moderate cytoplasmic staining. A cell may be classified 3+when it has intense cytoplasmic staining. If there are three thresholds defined, this score equals=(% 1+)+2* (% 2+)+3* (% 3+). Typically, there are three thresholds, so the H-score is between 0 and 300, where 300 represents 100% of cells being 3+.

In certain embodiments, nuclear expression of Ambra-1 and/or p62 is quantified using a nuclear algorithm (e.g., nuclear v9 algorithm). Such an algorithm may detect the nuclear staining for the individual cells in the nucleus and quantify their intensity.

For example, a nuclear intensity score derived from the average intensity of the staining of the nuclei (cellular average) may be determined according to the threshold intervals set in the algorithm macro. In one embodiment, nuclear staining may be classified as 0, 1+, 2+ and 3+based on nuclear staining intensity. A nucleus may be classified 0 when it has no nuclear staining. A nucleus may be classified 1+when it has weak nuclear staining. A nucleus may be classified 2+when it has moderate nuclear staining. A nucleus may be classified 3+when it has intense nuclear staining. If there are three thresholds defined, this score equals=(% 1+)+2* (% 2+)+3* (% 3+). Typically, there are 3 thresholds, the score is between 0 and 300, where 300 represents 100% of cells being 3+.

In certain embodiments, “weak”, “moderate” or “strong” staining of the cells is relative to levels of Ambra-1 and/or p62 characteristic of reference or normal tissue.

In certain embodiments, the method of determining expression of Ambra-1 and/or p62 comprises outputting, optionally on a computer, (i) an indication of the expression levels of Ambra-1 and/or p62 and (ii) this indicates whether the subject has an increased risk of progression and/or recurrence of the tumour.

In certain embodiments, an increased risk of progression and/or recurrence of the tumour means a 7-year metastasis-free (also known as “disease-free”) survival rate of less than 50%, for example less than 40%, for example less than 30%, for example less than 20%, for example less than 10%, for example less than 5%. The methods described herein allow subjects with SCC to be stratified into those more likely to develop progressive and/or recurrent tumours and those less likely to develop progressive and/or recurrent tumours.

Advantageously, the methods of the invention help to identify which subjects with SCC are most likely to benefit from treatment with a therapeutic agent. Typically, methods of the invention enable treatment with a therapeutic agent for a subject who would otherwise not have been eligible for treatment with a therapeutic agent.

### Reference Levels

In certain embodiments, the method for determining whether a subject with SCC has an increased risk of progression and/or recurrence of a tumour comprises comparing the expression of Ambra-1 and/or p62 in the one or more tissue samples obtained from the subject with one or more reference levels.

In some embodiments, the reference levels are levels of Ambra-1 and/or p62 expression that are characteristic of normal tissue. For example, reference levels of Ambra-1 and/or p62 may be obtained by determining the expression of Ambra-1 and/or p62 in a reference tissue.

In some embodiments, the expression levels of Ambra-1 and/or p62 in a reference tissue are determined by visual or automated assessment as described herein. In some embodiments, reference levels of Ambra-1 and/or p62 expression that are characteristic of normal tissue are obtained by determining expression levels in tissue samples obtained from one or more (e.g. a cohort) of healthy subjects.

In some embodiments, the reference tissue comprises normal tissue. In some embodiments, the normal tissue comprises tissue which does not include a primary carcinoma.

In some embodiments, the reference tissue (or levels obtained therefrom) is an internal reference (i.e. obtained from the subject). In some embodiments, the reference tissue is normal tissue obtained from a site adjacent to the primary carcinoma. In other embodiments, the reference tissue is obtained from a site of the subject which is remote from the primary carcinoma.

Thus, in some embodiments, the reference level is the level of expression of Ambra-1 and/or p62 in normal tissue. The reference tissue may be taken from normal epidermis and the reference level is a level of expression in the keratinocytes of the normal epidermis. The expression of Ambra-1 and/or p62 in the reference tissue, for example, to generate reference levels, can be determined using the methods described herein.

In preferred embodiments, the one or more reference levels are a predetermined cut-off value of Ambra-1 and/or p62 based on a H-score as described herein.

For example, the reference level may be a pre-determined cut-off value of Ambra-1 and/or p62 obtained from a cohort of SCC samples where subject outcome is already known. The cut-off value may be separately determined for normal epidermis, peritumoral epidermis, tumour mass and/or tumour growth front of a cohort of the SCC samples. The cut-off value may depend on the type of tissue being analysed.

In certain embodiments, the cut-off value is calculated using a ROC curve as described in the art.

In certain embodiments, the cut-off value of Ambra-1 and/or p62 is based on a H-score.

For example, the cut-off value of Ambra-1 expression in the tumour growth front may be a H-score of about 60. Typically, the H-score is 59.740.

In certain embodiments, a H-score of Ambra-1 is determined in the tumour growth front of one or more SCC samples obtained from the subject. If the H-score is below the cut-off value of about 60 (e.g., 59.740), the subject may have an increased risk of progression and/or recurrence of the tumour. Conversely, if the H-score is above the cut-off value of about 60 (e.g., 59.740), the subject may have a decreased risk of progression and/or recurrence of the tumour.

In addition or alternatively, the cut-off value of p62 expression in the peritumoral epidermis may be a H-score of about 20. Typically, the H-score is 20.010.

In certain embodiments, a H-score of p62 is determined in the peritumoral epidermis of one or more SCC samples obtained from the subject. If the H-score is below the cut-off value of about 20 (e.g., 20.010), the subject may have an increased risk of progression and/or recurrence of the tumour. Conversely, if the H-score is above the cut-off value of about 20 (e.g., 20.010), the subject may have a decreased risk of progression and/or recurrence of the tumour.

### Antibodies Against Ambra-1 or p62

In certain embodiments of the invention, antibodies against Ambra-1 are used to determine the expression of Ambra-1 in a tissue sample obtained from the subject. Similarly, antibodies against p62 may be used to determine the expression of p62 in the tissue obtained from the subject.

Antibodies against Ambra-1 or p62 include any polyclonal antibodies, any monoclonal antibodies, including chimeric antibodies, humanized antibodies, bi-specific antibodies and domains and fragments of monoclonal antibodies including Fab, Fab′, F(ab′)2, scFv, dsFv, ds-scFv, dimers, minibodies, diabodies, and multimers thereof. Monoclonal antibodies can be fragmented using conventional techniques. Monoclonal antibodies may be from any animal origin, including birds and mammals (e.g., human, murine, donkey, sheep, rabbit, goat, guinea pig, camel, horse, or chicken), transgenic animals, or from recombinant sources. Typically, the monoclonal antibodies against Ambra-1 or p62 are fully human. Monoclonal antibodies may be prepared using any methods known to those skilled in the art, including by recombination.

Typically, the antibody against Ambra-1 or p62 is isolated. An “isolated” antibody is an antibody that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, silver stain.

In certain embodiments, the antibody against Ambra-1 is a fragment that specifically binds Ambra-1.

In certain embodiments, the antibody against p62 is a fragment that specifically binds p62.

An “antibody fragment” is a portion of an intact antibody that includes an antigen binding site of the intact antibody and thus retaining the ability to bind Ambra-1 or p62 respectively.

Antibody fragments include:
(i) Fab fragments, having V_{L}, C_{L}, V_{H} and CH1 domains;
(ii) Fab′ fragments, which is a Fab fragment having one or more cysteine residues at the C-terminus of the CH1 domain;
(iii) Fd fragments having V_{H} and CH1 domains;
(iv) Fd′ fragments having V_{H} and CH1 domains and one or more cysteine residues at the C-terminus of the CH1 domain;
(v) Fv fragments having the V_{L} and V_{H} domains of a single arm of an antibody;
(vi) dAb fragments (Ward et al., Nature 341, 544-546 (1989)) which consist of a V_{H} domain);
(vii) isolated CDR regions, including any one or more of SEQ ID Nos 1 to 6, SEQ ID Nos 23 to 28 and SEQ ID Nos 41 to 46;
(viii) F(ab′)₂ fragments, a bivalent fragment including two Fab′ fragments linked by a disulphide bridge at the hinge region;
(ix) single chain antibody molecules (e.g. single chain Fv; scFv) (Bird et al, Science 242:423-426 (1988); and Huston et al., PNAS (USA) 85:5879-5883 (1988));
(x) “diabodies” with two antigen binding sites, comprising a heavy chain variable domain (VH) connected to a light chain variable domain (VL) in the same polypeptide chain (see, e.g., EP 404,097 and WO 93/11161);
(xi) “linear antibodies” comprising a pair of tandem Fd segments (VH-CH1-VH—CH1) which, together with complementary light chain polypeptides, form a pair of antigen binding regions (e.g. Zapata et al. Protein Eng. 8 (10): 1057-1062 (1995); and U.S. Pat. No. 5,641,870).

Typically, the antibody against Ambra-1 or p62 is a recombinant monoclonal antibody. A “recombinant monoclonal antibody” is an antibody or antibody fragment produced using recombinant antibody coding genes. In certain embodiments, the antibodies of the invention are generated from a human combinatorial antibody library (e.g. HuCAL, BioRad).

Typically, the antibody against Ambra-1 or p62 is a monovalent Fab or bivalent Fab fragment. A “bivalent Fab fragment” may be considered as equivalent to a F(ab′)2 fragment and formed via dimerization. For example, a bivalent Fab fragment is formed via dimerization of a synthetic double helix loop helix motif (dHLX) or a bacterial alkaline phosphatase (AP) domain. In certain embodiments, the antibody against Ambra-1 and/or the antibody against p62 comprises a dimerization domain sequence and one or more linker sequences.

In certain embodiments, the antibody against Ambra-1 or p62 is a recombinant monoclonal antibody fragment converted into an immunoglobulin (Ig) format. For example, when an Fc region is required, the variable heavy and light chain genes may be cloned into vectors with the desired constant regions and co-transfected for expression in mammalian cells using methods known to those skilled in the art. In certain embodiments, antibody fragments are converted to human IgA, IgE, IgG1, IgG2, IgG3, IgG3 or IgM.

In certain embodiments, the antibody against Ambra-1 or p62 is labelled with at least one epitope tag. Typical epitope tags include His6, Flag (e.g. DYKDDDDK, SEQ ID NO: 69), V5 (e.g. GKPIPNPLLGLDST, SEQ ID NO: 70), Strep (e.g. WSHPQFEK, SEQ ID NO: 71) and/or any combination thereof. Typically, the antibody against Ambra-1 and/or the antibody against p62 is a monovalent Fab or bivalent Fab fragment with one or more (e.g. two) epitopes. In certain embodiments, the antibody against Ambra-1 or p62 is conjugated to an enzyme and/or fluorescent label.

In certain embodiments, the antibody specifically binds to Ambra-1. In other words, the antibody against Ambra-1 may bind Ambra-1 with a binding dissociation equilibrium constant (K_{D}) of less than about 30 nM, less than about 20 nM, less than about 10 nm, less than about 1 nm or less than about 200 μm.

In certain embodiments, the antibody specifically binds to p62. In other words, the antibody against p62 may bind p62 with a binding dissociation equilibrium constant (K_{D}) of less than about 30 nM, less than about 20 nM, less than about 10 nm, less than about 1 nm or less than about 200 pm.

The skilled person would understand techniques for measuring binding strengths (e.g. koff-rate determination; ‘secondary screening’) include, for example, Bio-Layer Interferometry (e.g. using the Pall ForteBio Octet® System).

In certain embodiments, the antibody against Ambra-1 is available from a commercial supplier.

For example, the antibody against Ambra-1 may be AbCAM Ab69501 as described herein. Alternatively, the antibody against Ambra-1 may be BioRad AbD33473 as described herein. In certain embodiments, the antibody against Ambra-1 comprises the following heavy chain variable domain complementarity determining regions (CDRs):
(a) HCDR1 comprising the amino acid sequence of SEQ ID NO: 1;
(b) HCDR2 comprising the amino acid sequence of SEQ ID NO: 2; and/or
(c) HCDR3 comprising the amino acid sequence of SEQ ID NO: 3.

In certain embodiments, the antibody against Ambra-1 further comprises the following light chain variable domain CDRs:
(d) LCDR1 comprising the amino acid sequence of SEQ ID NO: 4
(e) LCDR2 comprising the amino acid sequence of SEQ ID NO: 5; and/or
(f) LCDR3 comprising the amino acid sequence of SEQ ID NO: 6.

As used herein, the term “Complementarity Determining Regions” (CDRs) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a CDR region can include amino acids from both a CDR region defined according to Kabat and a hypervariable loop.

Unless otherwise specified herein, numbering of amino acid residues in the Fc region or constant region is according to the EU numbering system, also called the EU index, as described in Kabat et al.

In certain embodiments, the antibody against Ambra-1 further comprises the following heavy chain variable domain framework regions (FRs):
(a) HCFR1 comprising the amino acid sequence of SEQ ID NO:7;
(b) HCFR2 comprising the amino acid sequence of SEQ ID NO:8;
(c) HCFR3 comprising the amino acid sequence of SEQ ID NO: 9; and/or
(d) HCFR4 comprising the amino acid sequence of SEQ ID NO: 10.

In certain embodiments, the antibody against Ambra-1 further comprises the following light chain variable domain FRs:
(e) LCFR1 comprising the amino acid sequence of SEQ ID NO:11;
(f) LCFR2 comprising the amino acid sequence of SEQ ID NO: 12;
(g) LCFR3 comprising the amino acid sequence of SEQ ID NO: 13; and/or
(h) LCFR4 comprising the amino acid sequence of SEQ ID NO: 14.

As used herein, “Framework regions (FRs)” are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues.

If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

In certain embodiments, the antibody against Ambra-1 further comprises an antibody variable domain comprising:
(a) a V_{H} sequence having at least 90%, at least 95%, at least 96%, at least 97%, at least 98%, at least 99% or more sequence identity to the amino acid sequence of SEQ ID NO: 15;
(b) a V_{L} sequence having at least 90%, at least 95%, at least 96%, at least 97%, at least 98%, at least 99% or more sequence identity to the amino acid sequence of SEQ ID NO 16; or
(c) a V_{H} sequence as in (a) and a V_{L} sequence as in (b).

In certain embodiments, the antibody against Ambra-1 comprises:
(d) a V_{H} sequence comprising SEQ ID NO: 15;
(e) a V_{L} sequence comprising SEQ ID NO: 16; or
(f) a V_{H} sequence as in (d) and a V_{L} sequence as in (e).

As used herein, “antibody variable domain” refers to the portions of the light and heavy chains of antibody molecules that include amino acid sequences of the CDRs (i.e., CDR1, CDR2, and CDR3) and the FRs (i.e., FR1, FR2, FR3 and FR4). V_{H} refers to the variable domain of the heavy chain. V_{L} refers to the variable domain of the light chain.

As used herein, “sequence identity” refers to a sequence having the specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by alignment software. In certain embodiments, the default parameters of the alignment software are used.

In certain embodiments, the antibody against Ambra-1 comprises a Fab fragment comprising the Fd chain of SEQ ID NO: 17; and/or the light chain of SEQ ID NO: 18. Typically, such antibodies further comprise one or more dimerization domain sequences, one or more linker sequences and/or one or more epitope tags as described herein.

In certain embodiments, the antibody against p62 is available from a commercial supplier. For example, the antibody against p62 may be AbCAM Ab96134 as described herein. Alternatively, the antibody against Ambra-1 may be BioRad AbD34907 or AbD34908 as described herein.

In certain embodiments, the antibody against p62 comprises the following heavy chain variable domain complementarity determining regions (CDRs):
(a) HCDR1 comprising the amino acid sequence of SEQ ID NO: 23 or 41;
(b) HCDR2 comprising the amino acid sequence of SEQ ID NO: 24 or 42; and/or
(c) HCDR3 comprising the amino acid sequence of SEQ ID NO: 25 or 43.

In certain embodiments, the antibody against p62 further comprises the following light chain variable domain CDRs:
(d) LCDR1 comprising the amino acid sequence of SEQ ID NO: 26 or 44;
(e) LCDR2 comprising the amino acid sequence of SEQ ID NO: 27 or 45; and/or
(f) LCDR3 comprising the amino acid sequence of SEQ ID NO: 28 or 46.

In certain embodiments, the antibody against p62 further comprises the following heavy chain variable domain framework regions (FRs):
(a) HCFR1 comprising the amino acid sequence of SEQ ID NO: 29 or 47;
(b) HCFR2 comprising the amino acid sequence of SEQ ID NO: 30 or 48;
(c) HCFR3 comprising the amino acid sequence of SEQ ID NO: 31 or 49; and/or
(d) HCFR4 comprising the amino acid sequence of SEQ ID NO: 32 or 50.

In certain embodiments, the antibody against p62 further comprises the following light chain variable domain FRs:
(e) LCFR1 comprising the amino acid sequence of SEQ ID NO: 33 or 51;
(f) LCFR2 comprising the amino acid sequence of SEQ ID NO: 34 or 52;
(g) LCFR3 comprising the amino acid sequence of SEQ ID NO: 35 or 53; and/or
(h) LCFR4 comprising the amino acid sequence of SEQ ID NO: 36 or 54.

In certain embodiments, the antibody against p62 further comprises an antibody variable domain comprising:
(a) a V_{H} sequence having at least 90%, at least 95%, at least 96%, at least 97%, at least 98%, at least 99% or more sequence identity to the amino acid sequence of SEQ ID NO: 37 or 55;
(b) a V_{L} sequence having at least 90%, at least 95%, at least 96%, at least 97%, at least 98%, at least 99% or more sequence identity to the amino acid sequence of SEQ ID NO: 38 or 56;
or
(c) a V_{H} sequence as in (a) and a V_{L} sequence as in (b).

In certain embodiments, the antibody against p62 comprises:
(d) a V_{H} sequence comprising SEQ ID NO: 37 or 55;
(e) a V_{L} sequence comprising SEQ ID NO: 38 or 56; or
(f) a V_{H} sequence as in (d) and a V_{L} sequence as in (e).

In certain embodiments, the antibody against p62 comprises a Fab fragment comprising the Fd chain of SEQ ID NO:39 and/or the light chain of SEQ ID NO:40. Alternatively, the antibody against p62 may comprise a Fab fragment comprising the Fd chain of SEQ ID NO: 57 and/or the light chain of SEQ ID NO: 58. Typically, such antibodies further comprise one or more dimerization domain sequences, one or more linker sequences and/or one or more epitope tags as described herein.

The invention provides use of antibodies (or aptamers) against Ambra-1 or p62 as described herein. Typically, the antibodies (or aptamers) against Ambra-1 or p62 are used in methods of determining whether a subject with SCC has an increased risk of tumour progression and/or recurrence as described herein.

The invention also provides antibodies (or aptamers) that compete for binding to Ambra-1 or p62 with antibodies (or aptamers) as described herein. Typically, competition assays are used to identify an antibody (or aptamer) that competes for binding to Ambra-1 or p62. In an exemplary competition assay, immobilized Ambra-1 or p62 is incubated in a solution comprising a first labelled antibody (or aptamer) that binds to Ambra-1 or p62 and a second unlabelled antibody (or aptamer) that is being tested for its ability to compete with the first antibody (or aptamer) for binding to Ambra-1 or p62. The second antibody (or aptamer) may be present in a hybridoma supernatant. As a control, immobilized Ambra-1 or p62 may be incubated in a solution comprising the first labelled antibody (or aptamer) but not the second unlabelled antibody (or aptamer). After incubation under conditions permissive for binding of the first antibody (or aptamer) to Ambra-1 or p62 excess unbound antibody (or aptamer) may be removed, and the amount of label associated with immobilized Ambra-1 or p62 measured.

If the amount of label associated with immobilized Ambra-1 or p62 is substantially reduced in the test sample relative to the control sample, then that indicates that the second antibody (or aptamer) is competing with the first antibody (or aptamer) for binding to Ambra-1 or p62 respectively. See, e.g., Harlow et al. Antibodies: A Laboratory Manual. Ch. 14 (Cold Spring Harbor Laboratory, Cold Spring Harbor, NY, 1988).

In certain embodiments, antibodies (or aptamers) that compete for binding to Ambra-1 or p62 bind to the same epitope (e.g., a linear or a conformational epitope) as the antibodies (or aptamers) described herein. Detailed exemplary methods for mapping an epitope to which an antibody binds are provided in Morris “Epitope Mapping Protocols,” in Methods in Molecular Biology Vol. 66 (Humana Press, Totowa, NJ, 1996).

Certain aspects of the present invention further provide isolated nucleic acids that encode any of the antibodies (or aptamers) described herein. Also provided is a vector (e.g., an expression vector) comprising the nucleic acid for expressing any of the antibodies (or aptamers) described herein. Also provided are host cells comprising the preceding nucleic acids and/or vectors.

Certain aspects of the present invention further provide immunoconjugates comprising any of the antibodies (or aptamers) described herein conjugated to one or more capture agents. As described herein, a capture agent typically comprises a binding and/or detection moiety (e.g. an enzyme and/or fluorescent label).

Certain aspects of the present invention further provide antibodies (or aptamers) that bind to the same epitope as the anti-Ambra-1 or p62 antibodies (or aptamers) as described herein.

In certain embodiments, the invention provides antibodies (or aptamers) that bind specifically to peptides near the carboxyl-terminus of human Ambra-1.

In certain embodiments, the invention provides antibodies (or aptamers) that bind to p62, wherein said antibodies (or aptamers) specifically bind near the carboxyl-terminus of human p62. In certain embodiments, the invention provides antibodies (or aptamers) that bind to p62, wherein said antibodies (or aptamers) specifically bind to a region comprising 8, 9, 10, 11, 12, 13, 14, 15, 16, 17, 18, 19, 20 or more of the amino acids shown in SEQ ID NO: 62.

As used herein, the term “epitope” means a protein determinant capable of specific binding to an antibody. Typically, an epitope comprises chemically active surface groupings of molecules such as amino acids or sugar side chains usually having specific three-dimensional structural and charge characteristics. The epitope may comprise amino acid residues directly involved in the binding and optionally additional amino acid residues that are not directly involved in the binding.

As used herein, an antibody (or aptamer) that “specifically” binds to an epitope refers to an antibody (or aptamer) that recognizes the epitope while only having little or no detectable reactivity with other portions of Ambra-1. Such relative specificity can be determined e.g. by competition assays, foot-printing techniques or mass spectrometry techniques as known in the art.

In certain embodiments, the epitope comprises peptide antigenic determinants within single peptide chains of Ambra-1. In certain embodiments, the epitope comprises conformational antigenic determinants comprising one or more contiguous amino acids on a particular chain and/or on spatially contiguous but separate peptide chains. In certain embodiments, the epitope comprises post-translational antigenic determinants comprising molecular structures (e.g. carbohydrate groups) covalently attached to Ambra-1.

The epitope may comprise any suitable number and/or type of amino acids, in any suitable position as defined herein. For example, the epitope may comprise about 3 to about 10 amino acids, typically about 3 to about 8 amino acids, in or more contiguous or non-contiguous locations with respect to the amino acid sequence of Ambra-1 as set forth in SEQ ID NO: 21, 22 and/or 63.

In certain embodiments, the invention provides antibodies (or aptamers) that bind to Ambra-1, wherein said antibodies (or aptamers) specifically bind to a region comprising 8, 9, 10, 11, 12, 13, 14, 15, 16, 17, 18, 19, 20 or more of the amino acids 1270 to 1298 of human Ambra-1 sequence shown in SEQ ID NO: 21. Typically, such antibodies (or aptamers) specifically bind to a region comprising amino acids 1280 to 1281 of SEQ ID NO: 21, amino acids 1294 to 1296 of SEQ ID NO: 21 and/or amino acids 1294 to 1297 of SEQ ID NO: 21.

In certain embodiments, the invention provides antibodies (or aptamers) that bind to Ambra-1, wherein said antibodies (or aptamers) specifically bind to a region comprising 8, 9, 10, 11, 12, 13, 14, 15 or more of CGGSSRGDAAGPRGEPRNR (SEQ ID NO: 22). Typically, such antibodies (or aptamers) specifically bind to a region comprising at least EPRN (SEQ ID NO: 67) or EPR (SEQ ID NO: 68) of Ambra-1.

In certain embodiments, the invention provides antibodies (or aptamers) that bind to Ambra-1, wherein said antibodies (or aptamers) specifically bind to a region comprising 8, 9, 10, 11, 12, 13, 14, 15 or more of DGGSSRGDAAGPRGEPRNR (SEQ ID NO: 64). Typically, such antibodies (or aptamers) specifically bind to a region comprising at least DG, EPRN (SEQ ID NO: 67) and/or EPR (SEQ ID NO: 68) of Ambra-1.

In certain embodiments, the invention provides antibodies (or aptamers) that bind to Ambra-1, wherein said antibodies (or aptamers) specifically bind to a region comprising HLLDGGSSR (SEQ ID NO: 65) and/or EPR (SEQ ID NO: 68) of Ambra-1.

In certain embodiments, the invention provides antibodies (or aptamers) that bind to Ambra-1, wherein said antibodies (or aptamers) specifically bind to a region comprising NHLLDGGSSR (SEQ ID NO: 66) and/or EPR (SEQ ID NO: 68) of Ambra-1.

In certain embodiments, the invention provides a method of labelling Ambra-1 and/or p62 in one or more tissue samples, the method comprising:
(a) contacting the tissue with an antibody against Ambra-1 and/or an antibody against p62 as described herein; and
(b) visualising the antibody against Ambra-1 and/or antibody against p62 in the tissue with a reagent that generates a detectable signal.

The tissue sample may comprise normal epidermis, peritumoral epidermis, tumour mass and/or tumour growth front of SCC tissue. Typically, the tissue sample is a biopsy, or a section thereof, obtained from a subject suffering from SCC.

In certain embodiments, a decrease or loss in the expression of Ambra-1 and/or p62 compared to reference levels may be determined (e.g. visualised) within the tissue. Such an expression pattern may be indicative of an increased risk of disease progression and/or recurrence of the tumour.

Alternatively, an increase or no significant difference in the expression of Ambra-1 and/or p62 compared to reference levels may be determined (e.g. visualised) within the tissue. Such an expression pattern may be indicative of a low risk of disease progression and/or recurrence of the tumour.

Aptly, automated or semi-quantitative immunohistochemistry is used to determine and/or quantify the expression pattern of Ambra-1 and/or p62 in the tissue sample as further described herein.

### Methods of Treatment

In certain embodiments, the invention provides methods of determining a treatment regime for a subject suffering from SCC comprising determining the expression of Ambra-1 and p62 as described herein.

Aptly, the method further comprises comparing the expression of Ambra-1 and p62 in the tissue with reference levels as described herein.

In certain embodiments, if there is increased or no significant difference (e.g. normal) expression of Ambra-1 and p62 as compared to one or more reference level(s) a normal recognized care pathway may be followed.

A “normal recognized care pathway”, as will be known to those skilled in the art, may mean that a wider excision of the scar left by excision of the primary carcinoma is carried out on the subject. The size of the wider excision will be determined by a clinician or surgeon, based on, for example, the Breslow depth of the primary carcinoma.

In certain embodiments, a normal recognized care pathway may comprise regular (e.g. every 3-12 months) clinical assessment of the subject for up to 5 years. The normal recognized care pathway may further comprise carrying out a staging CT scan on the subject, from the head to the pelvis, at the time of diagnosis. Thus, in some embodiments, the normal recognized care pathway may further comprise carrying out a sentinel lymph node biopsy.

In certain embodiments, a normal recognized care pathway may comprise topical chemotherapy, e.g. administration of fluorouracil (5-FU) or imiquimod to the skin and/or photodynamic therapy.

In certain embodiments, if expression of Ambra-1 and p62 is decreased or lost compared to the reference tissue or levels the subject may be treated with a systemic anti-cancer treatment regime.

In some embodiments, a systemic anti-cancer treatment regime comprises administering a therapeutic agent to the subject.

In some embodiments, the therapeutic agent is a chemotherapeutic agent. Any suitable chemotherapeutic agent may be administered to the subject. As used herein, a “chemotherapeutic agent” means any therapeutic agent useful for the treatment of cancer, including small molecules.

In some embodiments, the chemotherapeutic agent is a platinum-based chemotherapy. In some embodiments, the chemotherapeutic agent is selected from isotretinoin, interferon, retinoid, capecitabine, cisplatin and/or fluorouracil (5-FU). For example, the chemotherapeutic agent may be cisplatin and 5FU. In some embodiments, the chemotherapeutic agent is dacarbazine (DTIC) and/or temozolomide.

In some embodiments, the therapeutic agent is a biological agent.

In certain embodiments, the biological agent may be an anti-EGFR therapy e.g. cetuximab, panitumumab, nimotuzumab, zalutumumab, gefitinib, erlotinib, afatinib, lapatinib, neratinib and/or dacomitinib.

In certain embodiments, the biological agent may be a tyrosine kinase inhibitor e.g. gefetinib and/or imatinib. In certain embodiments, the biological agent is nilotinib or palbociclib.

In certain embodiments, the biological agent is an inhibitor of the 26S proteasome such as bortezomib.

In certain embodiments, the biological agent is an inhibitor of Human Papilloma Virus (HPV). In certain embodiments, the biological agent is an anti-BRAF therapy e.g., vemurafenib and/or dabrafenib.

In certain embodiments, the biological agent is a MEK inhibitor, e.g., trametinib and/or cobimetinib.

In certain embodiments, the biological agent is a combination anti-BRAF and anti-MEK therapy e.g., (i) dabrafenib and trametinib or cobimetinib or (ii) vemurafenib and trametinib or cobimetinib.

In certain embodiments, the biological agent is a checkpoint inhibitor. For example, the biological agent may be an anti-CTLA-4 therapy (e.g. ipilimumab). The biological agent may be an anti-PD-1 therapy (e.g. nivolumab, pembrolizumab or spartazumab). The biological agent may be an anti-PD-L1 therapy (e.g. atezolizumab).

In certain embodiments, the biological agent may be an anti-LAG3, anti-TIM-3, anti-TIGIT, anti-VISTA, anti-B7-H3, anti-KIR, anti-TGF-β, anti-OX40, anti-ICOS, anti-GITR, anti-4-1BB, anti-CD40, anti-IFO or anti-TLR therapy.

In certain embodiments, the biological agent is a CAR-T cell therapy.

In certain embodiments, the biological agent is a T-VEC vaccine (Imylgic), BCG vaccine, interferon, or IL-2.

In certain embodiments, the chemotherapeutic and/or biologic agent is used in combination with radiation.

In certain embodiments, the invention provides a method of treating a subject suffering from SCC comprising administering a therapeutic agent to a subject identified as having decreased or loss of expression of Ambra-1 and/or p62 following the methods described herein.

Ideally, a subject identified as having an increased risk of tumour progression and/or recurrence is treated as soon as possible to minimize the chances of development of the tumour. Thus, in some embodiments the method or treatment regime is for preventing, inhibiting or delaying tumour progression and/or recurrence or decreasing the risk of tumour progression and/or recurrence in the subject.

In some embodiments, a subject is treated immediately or shortly after being identified as having an increased risk of tumour progression and/or recurrence.

In some embodiments, treatment with the therapeutic agent is carried out after surgery to excise the primary carcinoma.

In some embodiments, a method of treatment or a treatment regime may further include one or more of: intensified imaging (e.g. CT scan, PET, MRI, X-ray) of the subject; discussion and/or offering of, or carrying out, a sentinel lymph node biopsy; partial or complete lymphadenectomy; inclusion of the subject in clinical trials; and radiation therapy.

In some embodiments, a therapeutic agent is administered to the subject no more than 12 weeks, no more than 10 weeks, no more than 6 weeks, no more than 4 weeks, no more than 2 weeks or no more than 1 week after the subject is identified as having a decrease or loss of expression of Ambra-1 and/or increased expression of p62 in the tissue sample. Non-limiting routes of administration of the therapeutic agent include by oral, intravenous, intraperitoneal, subcutaneous, intramuscular, topical, intradermal, intranasal or intrabronchial administration (for example as effected by inhalation). In some embodiments, the therapeutic agent is administered parenterally, e.g., intravenously. Common modes of administration by which the therapeutic agent may be administered include, for example, administration as a bolus dose or as an infusion over a set period.

A therapeutic agent may be administered in an amount effective to prevent, inhibit or delay the development of tumour progression and/or recurrence. Suitable doses and dosage regimes for a given subject and therapeutic agent can be determined using a variety of different methods, such as body-surface area or body weight, or in accordance with specialist literature and/or individual hospital protocols. Doses may be further adjusted following consideration of a subject's neutrophil count, renal and hepatic function, and history of any previous adverse effects to the therapeutic agent. Doses may also differ depending on whether a therapeutic agent is used alone or in combination.

The skilled person will recognize that further modes of administration, dosages of therapeutic agents and treatment regimens can be determined by the treating physician according to methods known in the art.

### EXAMPLES

### Example 1-Analysis of Powered Cohort of Primary cSCC Tumours

A retrospective (pre-2006) formalin-fixed paraffin-embedded (FFPE) cohort of 118 primary cSCC tumours derived from Addenbrookes Hospital, Cambridge of differing differentiation status (including 47 which reoccurred or metastasized) was analysed as part of this study.

TABLE 1
AMBRA/p62 biomarker discovery cSCC cohort
Addenbrookes Hospital Cambridge cSCC Cohort
Non-metastatic Primary Tumours

| | |
|---|---|
| Well Differentiated | 23 |
| Moderately Differentiated | 26 |
| Poorly Differentiated | 22 |

Metastatic /Recurrent Tumours

| | |
|---|---|
| Well Differentiated | 14 |
| Moderately Differentiated | 20 |
| Poorly Differentiated | 13 |

Staining of cSCC tumour cohorts for AMBRA1 and p62 expression was performed via automated immunohistochemistry, conducted by the Department of Pathology, Royal Victoria Infirmary, Newcastle Upon Tyne Hospitals NHS Foundation Trust, Newcastle upon Tyne, UK. All slides were stained with a primary antibody for the protein of interest and counterstained with 3,3′-diaminobenzidine (DAB) and Haemalum. Stained slides then had 40× magnification images taken using the Aperio AT2 Slide Scanner (Leica Biosystems, UK). The digital images of these tumours were then subject to AMBRA1 and p62 expression quantification as described below.

Anti-Ambra-1 and p62 antibodies and staining protocols used in this study are as previously described in WO 2020/225548 A1.

### Example 2-Development of a Digital Quantification Method to Analyse AMBRA1 or p62 Expression in the Growth Front, Tumour Mass, Peritumoural or Normal Epidermal Environment of Primary cSCCs

Automated immunohistochemistry for the expression of AMBRA1 and p62 was performed in all tissue sections derived from Cambridge Addenbrookes Hospital discovery cohort of cSCCs. Following the acquisition of digital images (40× magnification), four key regions of interest were identified (FIG. 1):
the ‘normal epidermis,’ defined as an epidermal region distant to the primary tumour
the peritumoural epidermis, defined as the epidermis directly alongside the primary tumour
the ‘tumour mass,’ defined as the principle tumour area (and not at the deepest aspect of the tumour or displaying an invasive phenotype)
tumour growth front,′ defined as the tumour regions at the deepest aspect or the region displaying a invasive phenotype.

The normal epidermis served as an internal control for AMBRA1 and p62 expression in keratinocytes in homeostatic conditions. The ‘peritumoural epidermis,’ on the other hand, represented keratinocytes that had either been exposed to the same tumorigenic field effect as the cells that had become transformed and/or cells that were subjected to the same signalling environment as the tumour. As such, keratinocytes in this environment are a good representation of cells that, whilst not fully functioning under homeostatic conditions, have not undergone full carcinogenic transformation. The area of the tumour growth front was analysed independently of cellular expression in the tumour mass as cells in this region had likely been through several additional mutagenic events, acquired pro-survival mechanisms and were beginning to exhibit a metastatic phenotype.

Digital images were annotated using the annotating tool within the Aperio ImageScope software (V12.4.2.5010, Leica Biosystems, UK), marking 2×1.00 mm² square boxes each for the normal and peritumoural epidermis and 5 representative areas/1.00 mm² square boxes each for the tumour mass and tumour growth front (FIG. 1). The number of square boxes used for each region was determined based on the observed variability within primary cSCC tumours. As there was little or no variability between the normal epidermis and peritumoural epidermis only two 1.00 mm² square boxes, one either side of the site of the primary tumour were annotated. However, since qualitative analysis of both AMBRA1 and p62 showed a high degree of intratumoural variability within primary cSCC tumours, multiple representative areas (×5) were selected to best encompass this variability. Further annotation was then undertaken within each box of interest ensuring all keratinocytes within recognisable epidermal structures in normal and peritumoural epidermal regions and all tumour cells for the annotated tumour mass and tumour growth front regions were captured (FIG. 1).

Following this, all annotated areas of interest were subjected to digital H score analysis using Aperio ImageScope software analysis with a pre-optimised cytoplasmic or nuclear algorithm. The H score from each representative area of interest defined the level of staining intensity of either AMBRA1 or p62 which was used to derive the mean H score value and correlate with clinical follow up data.

### Example 3-Loss of Cytoplasmic AMBRA1 Expression at the cSCC Tumour Growth Front, in Combination with Loss of Cytoplasmic, Peritumoural Epidermal p62 Expression is a Prognostic Biomarker for cSCC Disease Progression

ROC curve analysis was used to define the prognostic potential of cytoplasmic AMBRA1 or nuclear or cytoplasmic p62 expression for cSCC, where the greatest potential is given by the area under the curve (AUC) value, closest to 1.00.

ROC curve analysis of all primary cSCC tumours within the Cambridge cohort revealed cytoplasmic AMBRA1 expression in the tumour growth front region with an AUC value of 0.5622 and cytoplasmic p62 expression in the peritumoural epidermis with an AUC of 0.6178 as the regions with greatest prognostic potential (FIG. 2). Within the tumour growth front region, a mean AMBRA1 H-score value of <59.740 (FIG. 2A) had the highest sensitivity and specificity while for cytoplasmic p62 an H-score value <20.010 (FIG. 2B) had the highest sensitivity and specificity for identifying primary cSCC tumours at risk of disease recurrence or metastasis.

ROC curve analysis of cytoplasmic AMBRA1 expression in the peritumoural epidermis, and the tumour mass and growth front was also undertaken in sub-cohorts of well, moderately and poorly differentiated primary cSCC tumours. Regardless of cellular differentiation status, cytoplasmic AMBRA1 expression in the tumour growth front region was consistently the region with the highest AUC value.

However, for expression of p62, ROC curve analysis in sub cohorts of well, moderately or poorly differentiated cSCCs identified different regions in which cytoplasmic or nuclear expression corresponded with the highest prognostic potential. For well-differentiated cSCCs loss of cytoplasmic p62 expression in the peritumoural epidermis gave the highest prognostic potential, while in moderately differentiated cSCCs, loss of cytoplasmic p62 expression in the tumour growth front or in poorly differentiated cSCCs the increase in nuclear p62 expression in the peritumoural epidermis gave the highest prognostic potential.

Taken together, these data demonstrate that regardless of tumour differentiation status, the loss of cytoplasmic AMBRA1 expression in the tumour growth front region and the loss of cytoplasmic p62 expression in the peritumoural epidermis have the highest potential to identify cSCCs at risk of disease recurrence or metastasis.

Having determined the optimal expression levels of AMBRA1 in the tumour growth front and p62 in the peritumoural epidermis with greatest prognostic potential, survival curve analysis was then performed to determine the potential or either marker or the combined prognostic potential of these two biomarkers. Initial analysis based on cytoplasmic AMBRA1 expression in the tumour growth front region alone and a cut off H-score of <59.740 to define a high-risk tumour revealed no significant difference in predicting disease progression in high or low risk subsets; 73.59% of tumours defined as low-risk and 57.58% of tumours defined as high-risk had no disease event within 60 months, collectively indicating the unsuitability of AMBRA1 expression in the tumour growth front alone as a prognostic biomarker for cSCC.

Additional Kaplan-Meier survival analysis of AMBRA1 expression in the tumour growth front of sub-cohorts of either well, moderately or poorly differentiated cSCCs also demonstrated the inability of this single marker to identify low and high-risk tumour subsets.

Kaplan-Meir survival analysis based on cytoplasmic p62 expression in the peritumoural epidermis alone and a cut off mean H-score of <20.01 to define a high-risk subset was also unable to define high risk cSCC subsets; 72.72% of tumours defined as low-risk and 69.57% tumours defined as high-risk had no disease event within 60 months, again, collectively indicating the inability of p62 expression in the peritumoural epidermis alone as a prognostic biomarker for cSCC.

Similarly, to analysis of AMBRA1 in the tumour growth front of sub cohorts of primary cSCCs with differing differentiation status, Kaplan-Meier survival analysis based on both cytoplasmic and nuclear p62 expression was performed in well, moderately and poorly differentiated tumours. Whilst cytoplasmic p62 expression in the peritumoural epidermis of well and cytoplasmic p62 expression in the tumour growth front of moderately differentiated primary cSCCs had the greatest prognostic potential, neither marker alone was able to significantly distinguish between high and low risk tumour subsets. Nuclear p62 expression in the peritumoural epidermis, on the other hand was able to distinguish disease recurrence or metastasis in tumour subsets of poorly differentiated cSCCs.

Given the limited ability of AMBRA1 expression in the growth front of cSCCs or the expression of cytoplasmic p62 in the peritumoural epidermis as single biomarkers, their capacity as combined prognostic biomarkers was evaluated in the entire Cambridge cohort of poorly, moderately or well differentiated localised or recurrent/metastatic cSCCs (FIG. 3) as well as in individual sub cohorts of well, moderately or poorly differentiated tumours. A tumour with a cytoplasmic AMBRA1 H-score in the growth front region of <59.74 and a mean cytoplasmic p62 H score in the peritumoural epidermis of <20.010, was defined as being at high risk of a disease recurrence/metastasis, while a cytoplasmic AMBRA1 H-score in the tumour growth front ≥59.74 and/or a mean cytoplasmic p62 H score in the peritumoural epidermis of ≥20.010 was categorised as low risk.

Resultant Kaplan-Meier survival curve analysis revealed dual AMBRA1 expression in the tumour growth front and p62 expression in the peritumoural epidermis significantly distinguished between high and low risk tumour subsets (P<0.05, FIG. 3); 78.57% of patients with low-risk tumours were disease free at 60 months compared to only 46.15% of patients with high-risk tumours. Furthermore, as a biomarker, the combined AMBRA1 tumour growth front and peritumoural epidermal p62 expression predicted recurrence/metastasis of primary cSCCs with a hazard ratio of 4.421 (95% Cl 1.322-14.790), a negative predictive value of 75.38%, a positive predictive value of 53.85%, an assay specificity of 89.09% and sensitivity of 30.43%.

In summary these data suggest the combined expression of cytoplasmic AMBRA1 in the tumour growth front and cytoplasmic p62 expression in the peritumoural epidermis as a novel prognostic biomarker for cSCC disease recurrence/metastasis.

### Example 4-Loss of Cytoplasmic AMBRA1 Expression at the cSCC Tumour Growth Front, in Combination with Loss of Cytoplasmic, Peritumoural Epidermal p62 Expression is a Prognostic Biomarker for Metastasis in Moderately and Poorly Differentiated cSCC Tumours

Having established the combined expression of cytoplasmic AMBRA1 in the tumour growth front and cytoplasmic p62 in the peritumoural epidermis is able to predict cSCC recurrence and metastasis, further sub cohort analysis was undertaken to evaluate the improved potential of this combined biomarker to predict metastasis with in well, moderately, poorly or moderately/poorly differentiated cSCCs.

The prognostic ability of AMBRA1 tumour growth front and p62 peritumoural epidermal expression was significantly improved in moderately (P<0.01, FIG. 4) and poorly differentiated cSCCs (P<0.05, FIG. 5) by removal of the recurrent tumour subsets. Specifically, metastasis at 60 months did not occur in 70.37% patients with low risk moderately differentiated primary cSCC tumours while all patients with high-risk tumours developed metastasis by 60 months (FIG. 4). 82.35% of patients with poorly differentiated low risk primary cSCC tumours on the other hand did not develop metastatic disease at 60 months, while 66.67% of patients with high-risk tumours developed metastasis by 60 months (FIG. 5). Collectively these data suggest combined AMBRA1 tumour growth front and peritumoural epidermal p62 expression as a viable prognostic biomarker of metastasis in moderately or poorly differentiated cSCCs.

Clinically primary cSCCs are rarely defined as moderately or poorly differentiated and are more commonly categorised as moderately/poorly differentiated tumours. Further validating the potential for combined AMBRA1 tumour growth front and peritumoural epidermal p62 expression as a prognostic biomarker for disease metastasis in combined moderately/poorly differentiated cSCCs, Kaplan-Meier survival curve analysis revealed a significant increase in the ability to predict metastasis in these tumours (P<0.0001, FIG. 6); 75.00% patients with low risk tumours did not develop metastasis at 60 months compared to 85.71% of patients with high risk tumours that developed metastatic disease. Furthermore as a biomarker, the combined AMBRA1 tumour growth front and peritumoural epidermal p62 expression predicts metastasis of moderately/poorly differentiated cSCCs with a hazard ratio of 30.07 (95% Cl 5.48-165), a negative predictive value of 75.00%, a positive predictive value of 85.71%, an assay specificity of 97.06% and sensitivity of 35.29%. Collectively this highlights the power and potential clinical impact of combined AMBRA1 tumour growth front and peritumoural epidermal p62 expression as a novel prognostic biomarker for moderately/poorly differentiated cSCC.

### DISCUSSION

The ideal biomarker for any disease should leave little to no interpretation by a histopathologist and as such, ROC curve analysis was undertaken to better assess the prognostic potential of either AMBRA1 or p62 expression in any defined cSCC tumour region (Kamarudin et al., 2017). ROC curve analysis was conducted on all primary cSCC tumours regardless of differentiation status, to limit potential bias and misinterpretation by histopathologists of influencing factors such as tumour differentiation. However, ROC curve analysis was nevertheless performed in sub-cohorts of well, moderately and poorly differentiated cSCCs to evaluate the potential of tumour differentiation status as a prognostic variable.

Results revealed loss of cytoplasmic AMBRA1 expression in the tumour growth front region and loss of cytoplasmic p62 in the peritumoural epidermis of region in all primary cSCC tumours had the highest level of prognostic potential, with an AUC of 0.5522 and 0.6352 respectively (FIG. 2).

This result contradicts a sizeable amount of previously published work demonstrating the upregulation of autophagy is vital for the initiation and survival of metastatic cancer cells. This increase in active autophagy has been implicated in the scavenging and reuse of vital cell nutrients, the process of epithelial-to-mesenchymal transition, resistance to anoikis, cell motility, immune detection avoidance and the rearrangement of focal adhesion complexes (Kenific et al., 2010, Li et al., 2013, Fung et al., 2008, Kadandale et al., 2010, Sharifi et al., 2016, Akalay et al., 2013), suggesting that the loss of the pro-autophagy protein AMBRA1 is illogical. Noteworthy, was the fact that a total loss of AMBRA1 expression was not observed in the tumour growth front of cSCC tumours suggesting the maintenance of some automatic activity. Given the recent suggestion that AMBRA1 is a critical regulator of keratinocyte differentiation (Cosgarea et al., 2021), this suggests loss of expression is therefore more representative of reduced cell differentiation capacity and the preservation of a more stem cell state, another vital component of cancer cell metastasis (Chaffer and Weinberg, 2011). Collectively the level of AMBRA1 expression likely demonstrates the retention of some autophagy capacity and a more stem like cancer cell state which together facilitate and promote cSCC metastasis.

Interestingly, and in contrast to all other studies which have explored the potential for p62 as a biomarker in cancer tumourigenesis or progression, the present study in cSCC suggests the prognostic potential for p62 expression is derived from its reduced expression in the peritumoural epidermis. Supporting this notion, p62 has been implicated in multiple tumour microenvironment reprogramming events, such lipid redistribution and metabolic reprogramming of adipose cells and Nrf2 activation of cancer-associated fibroblasts, allowing cancer tumourigenesis through an altered ECM (Reina-Campos et al., 2018, Kang et al., 2021). Additionally, recent work has demonstrated a correlation between breast cancer cell motility and suppressed p62 expression (Tan et al., 2018), suggesting the observed reduction in peritumoural epidermal p62 expression may allow early cSCC cell invasion due to a reduction in epidermal integrity. However, studies examining the specific role of p62 in the epithelial of tumour microenvironments is currently lacking, making it difficult to define the precise underlying mechanism underpinning the observed prognostic potential.

Taken together data from the present study suggest that individually AMBRA1 and p62 are not viable prognostic biomarkers for cSCC recurrence/metastasis. However, the combination of AMBRA1 expression in the tumour growth front with the cytoplasmic expression of p62 in the peritumoural epidermis was able to significantly distinguish high and low risk tumour subsets at risk of disease recurrence/metastasis independently of tumour differentiation status (FIG. 3, P<0.05, HR 4.421 (1.322 to 14.91).

Furthermore the prognostic potential of combined AMBRA1 expression in the tumour growth front and cytoplasmic expression of p62 in the peritumoural epidermis is further strengthened when removing recurrent primary tumours and stratifying for the risk of metastasis in moderately and/or poorly differentiated cSCC tumours (FIG. 4 for moderately differentiated sub cohort analysis, P<0.01, HR 21.080 (95% Cl 2.845-156.000), FIG. 5 for poorly differentiated sub cohort analysis, P<0.05, HR 25.260 (95% Cl 1.381-463.000), and FIG. 6 for moderately and poorly differentiated sub cohort analysis, P<0.0001, HR 30.070 (95% Cl 5.480-165.000).

A 40-gene biomarker test produced by Castle Bioscience is the most extensively studied biomarker to date for cSCC (Wysong et al., 2020). Comparing hazard ratios with the combined use of AMBRA1 and p62 in moderately/poorly differentiated cSCCs, it is clear to see use of AMBRA1/p62 outperforms the 40-gene test at a three-fold rate, with hazard ratios of 30.070 and 9.55 respectively. The positive predictive value (PPV) of 75% and the assay specificity of 97% for combined AMBRA/p62 expression are also significantly higher than the PPV or assay specificity for the 40 gene test. Furthermore, the negative predictive value of 86% for combined AMBRA1/p62 expression as a prognostic biomarker for metastatic development is comparable to the value of 91% reported for the 40 gene test. Assay sensitivity for combined AMBRA1/p62 expression is also comparable to that of the Castle Biosciences 40 gene test, with a value of 35.29%, which outperforms the Class 2B classification of the test at 28.80%. Additionally, the use of AMBRA1/p62 as an IHC based prognostic marker will fit seamlessly into current diagnostic procedures for cSCC, not requiring additional tissue as it can be performed using a serial FFPE tissue section and will easily be able to be developed as a rapid digital machine learning based test in line with the move of pathology services to a more digital approach.

Overall, the combined AMBRA1 expression in the tumour growth front and cytoplasmic expression of p62 in the peritumoural epidermis thus defines a novel prognostic biomarker able to predict disease recurrence/metastasis of cSCCs regardless of differentiation status, and more accurately as a prognostic biomarker of disease metastasis in moderately/poorly differentiated cSCCs.

The reader's attention is directed to all papers and documents which are filed concurrently with or before this specification in connection with this application and which are open to public inspection with this specification, and the contents of all such papers and documents are incorporated herein by reference.

1.-17. (canceled)

18. A method of treating a subject suffering from squamous cell carcinoma (SCC) comprising administering a therapeutic agent to the subject, wherein the subject has been identified as having decreased or loss of expression of Ambra-1 and p62 in one or more tissue sample(s) by determining the expression of Ambra-1 and p62 in one or more tissue samples obtained from the subject, wherein the tissue sample(s) comprise a primary tumour and tissue overlying the primary tumour, wherein a decrease or loss in the expression of Ambra-1 and a decrease or loss in the expression of p62 in the tissue sample(s) compared to one or more reference level(s) is indicative of an increased risk of progression and/or recurrence of the tumour.

19. The method of claim 18, wherein the therapeutic agent is a chemotherapeutic agent, optionally a chemotherapeutic agent selected from Dacarbazine (DTIC), Temozolomide, Nab-paclitaxel, Paclitaxel, Carmustine (BCNU), Cisplatin, Carboplatin, Vinblastine, interleukin 2, interferon alpha, antibodies and B-Raf inhibitors.

20. The method of claim 18, wherein the therapeutic agent is a biological agent, optionally a biological agent selected from:
(a) an anti-CTLA-4 therapy e.g. ipilimumab;
(b) an anti-PD-1 therapy e.g. nivolumab, pembrolizumab or spartalizumab;
(c) an anti-PD-L1 therapy e.g. atezolizumab;
(d) an anti-EGFR therapy, e.g., cetuximab, panitumumab, nimotuzumab, zalutumumab, gefitinib, erlotinib, afatinib, lapatinib, neratinib and/or dacomitinib);
(e) an anti BRAF therapy, e.g., vemurafenib and/or dabrafenib;
(f) a MEK inhibitor, e.g., trametinib and/or cobimetinib; and/or
(g) any combination thereof.

21. The method of claim 18, wherein the subject, prior to identification, was ineligible for therapeutic agent treatment.

22. The method of claim 18, wherein expression of Ambra-1 and/or p62 is determined in the cytoplasm of cells within the tissue.

23. The method of claim 18, wherein:
(i) the expression of Ambra-1 is determined in the tumour growth front of the primary tumour; and/or
(ii) the expression of p62 is determined in the peritumoral epidermis overlying the primary tumour.

24. The method of claim 18, wherein the primary tumour is moderately and/or poorly differentiated.

25. The method of claim 18, wherein determining the expression of Ambra-1 and/or p62 in the tissue sample(s) comprises contacting the tissue sample(s) with a ligand specific for Ambra-1 and/or p62.

26. The method of claim 25, wherein:
(i) the ligand specific for Ambra-1 is an antibody, optionally wherein the antibody specifically binds to the region EPRN, HLLDGGSSR and/or NHLLDGGSSR of human Ambra-1 (SEQ ID NO: 21); and/or
(ii) the ligand specific for p62 is an antibody, optionally wherein the ligand specifically binds to a protein having the sequence shown in SEQ ID NO: 62.

27. The method of claim 18, wherein the expression of Ambra-1 and/or p62 is determined by automated or semi-automated immunohistochemistry.

28. The method of claim 18, wherein the expression of Ambra-1 and/or p62 is scored on the basis of the intensity and/or proportion of positive cells in the tissue sample(s).

29. The method of claim 18, wherein the reference levels are a predetermined cut-off value of Ambra-1 and/or p62 based on a H-score, wherein optionally a H-score of about 60 or less within a tumour growth front of the primary tumour is indicative of a decrease or loss in the expression of Ambra-1 and/or wherein optionally a H-score of about 20 or less within peritumoral epidermis overlying the primary tumour is indicative of a decrease or loss in the expression of p62.

30. The method of claim 18, wherein the tissue sample(s) are a biopsy, or one or more sections thereof, obtained from the subject, and/or the SCC is cutaneous SCC (cSCC), and/or the subject is suffering from AJCC T1 or T2 SCC.

31. A method of determining whether a subject has an increased risk of progression and/or recurrence of squamous cell carcinoma (SCC), the method comprising:
contacting a tissue sample comprising a primary tumour and tissue overlying the primary tumour obtained from a subject with a first ligand specific for Ambra-1 comprising the sequence shown in SEQ ID NO: 21, the first ligand comprising an anti-Ambra-1 antibody, wherein the presence of Ambra-1 creates an Ambra-1 ligand complex;
contacting the tissue sample comprising the primary tumour and tissue overlying the primary tumour with a second ligand specific for p62 comprising the sequence shown in SEQ ID NO: 61, wherein the presence of p62 creates a p62 ligand complex; and,
detecting a co-occurrence of a decrease in the expression of Ambra-1 having the sequence shown in SEQ ID NO: 21, and a decrease in the expression of p62 having the sequence shown in SEQ ID NO: 61 in the tissue sample comprising the primary tumour and tissue overlying the primary tumour, or a loss of expression of Ambra-1 and a loss of expression of p62 in the tissue sample by detecting and/or quantifying the Ambra-1 ligand complex and the p62-ligand complex; and
predicting an increased risk of progression and/or recurrence of squamous cell carcinoma (SCC) if there is a co-occurrence of a decrease in the expression of Ambra-1 and a decrease in the expression of p62 in the tissue sample compared to one more reference level(s).

32. The method of claim 31, wherein the expression levels of Ambra-1 and p62 are determined by visual or automated assessment.

33. The method of claim 32, further comprising outputting on a computer (i) an indication of the expression levels of Ambra-1 and p62 and (ii) this indicates whether the subject has an increased risk of progression and/or recurrence of the tumour.


SEQUENCE LISTING (71 sequences)

SEQ ID NO: 1 - protein, 5 aa, synthetic construct
  REGION 1..5: note = HCDR1 of the anti-Ambra-1 antibody AbD33473
SYWIH

SEQ ID NO: 2 - protein, 17 aa, synthetic construct
  REGION 1..17: note = HCDR2 of the anti-Ambra-1 antibody AbD33473
TIFPSRSYTTYSPSFQG

SEQ ID NO: 3 - protein, 13 aa, synthetic construct
  REGION 1..13: note = HCDR3 of the anti-Ambra-1 antibody AbD33473
DTPSTALKSPFDY

SEQ ID NO: 4 - protein, 13 aa, synthetic construct
  REGION 1..13: note = LCDR1 of the anti-Ambra-1 antibody AbD33473
SGSSSNIGYNYVY

SEQ ID NO: 5 - protein, 7 aa, synthetic construct
  REGION 1..7: note = LCDR2 of the anti-Ambra-1 antibody AbD33473
ENNKRPS

SEQ ID NO: 6 - protein, 11 aa, synthetic construct
  REGION 1..11: note = LCDR3 of the anti-Ambra-1 antibody AbD33473
SSWDSHSNSYV

SEQ ID NO: 7 - protein, 30 aa, synthetic construct
  REGION 1..30: note = HCFR1 of the anti-Ambra-1 antibody AbD33473
EVQLVQSGAEVKKPGESLKISCKGSGYSFS

SEQ ID NO: 8 - protein, 14 aa, synthetic construct
  REGION 1..14: note = HCFR2 of the anti-Ambra-1 antibody AbD33473
WVRQMPGKGLEWMG

SEQ ID NO: 9 - protein, 32 aa, synthetic construct
  REGION 1..32: note = HCFR3 of the anti-Ambra-1 antibody AbD33473
QVTISADKSISTAYLQWSSLKASDTAMYYCAR

SEQ ID NO: 10 - protein, 11 aa, synthetic construct
  REGION 1..11: note = HCFR4 of the anti-Ambra-1 antibody AbD33473
WGQGTLVTVSS

SEQ ID NO: 11 - protein, 22 aa, synthetic construct
  REGION 1..22: note = LCFR1 of the anti-Ambra-1 antibody AbD33473
DIVLTQPPSVSGAPGQRVTISC

SEQ ID NO: 12 - protein, 15 aa, synthetic construct
  REGION 1..15: note = LCFR2 of the anti-Ambra-1 antibody AbD33473
WYQQLPGTAPKLLIY

SEQ ID NO: 13 - protein, 32 aa, synthetic construct
  REGION 1..32: note = LCFR3 of the anti-Ambra-1 antibody AbD33473
GVPDRFSGSKSGTSASLAITGLQAEDEADYYC

SEQ ID NO: 14 - protein, 12 aa, synthetic construct
  REGION 1..12: note = LCFR4 of the anti-Ambra-1 antibody AbD33473
FGGGTKLTVLGQ

SEQ ID NO: 15 - protein, 122 aa, synthetic construct
  REGION 1..122: note = VH domain of the anti-Ambra-1 antibody AbD33473
EVQLVQSGAEVKKPGESLKISCKGSGYSFSSYWIHWVRQMPGKGLEWMGTIFPSRSYTTYSPSFQGQVTISADKSISTAYLQWSSLKASDTAMYYCARDTPSTALKSPFDYWGQGTLVTVSS

SEQ ID NO: 16 - protein, 112 aa, synthetic construct
  REGION 1..112: note = VL domain of the anti-Ambra-1 antibody AbD33473
DIVLTQPPSVSGAPGQRVTISCSGSSSNIGYNYVYWYQQLPGTAPKLLIYENNKRPSGVPDRFSGSKSGTSASLAITGLQAEDEADYYCSSWDSHSNSYVFGGGTKLTVLGQ

SEQ ID NO: 17 - protein, 224 aa, synthetic construct
  REGION 1..224: note = Fd chain (VH domain and constant domain) of the Fab region of the anti-Ambra-1 antibody AbD33473
EVQLVQSGAEVKKPGESLKISCKGSGYSFSSYWIHWVRQMPGKGLEWMGTIFPSRSYTTYSPSFQGQVTISADKSISTAYLQWSSLKASDTAMYYCARDTPSTALKSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 18 - protein, 215 aa, synthetic construct
  REGION 1..215: note = light chain (VL domain and constant domain) of the Fab region of the anti-Ambra-1 antibody AbD33473
DIVLTQPPSVSGAPGQRVTISCSGSSSNIGYNYVYWYQQLPGTAPKLLIYENNKRPSGVPDRFSGSKSGTSASLAITGLQAEDEADYYCSSWDSHSNSYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 19 - DNA, 2085 bp, synthetic construct
  misc_feature 1..2085: note = Fd chain of the Fab region and tags (alkaline phosphatase dimerization domain sequence (AP), FLAG tag (DYKDDDDK) and His6 tag of the anti-Ambra-1 antibody AbD33473
  source 1..2085: mol_type = other DNA
gaagtgcaattggtgcagagcggtgcggaagtgaaaaaaccgggcgaaagcctgaaaattagctgcaaaggctccggatatagcttctcttcttactggatccattgggtgcgccagatgccgggcaaaggtctcgagtggatgggcactatcttcccgtctcgtagctacaccacttatagcccgagctttcagggccaggtgaccattagcgcggataaaagcatcagcaccgcgtatctgcaatggagcagcctgaaagcgagcgataccgcgatgtattattgcgcgcgtgacactccgtctactgctctgaaatctccgttcgattactggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 20 - DNA, 645 bp, synthetic construct
  misc_feature 1..645: note = Light chain of the Fab region of the anti-Ambra-1 antibody AbD33473
  source 1..645: mol_type = other DNA
gatatcgtgctgacccagccgccgagcgtgagcggtgcaccgggccagcgcgtgaccattagctgtagcggcagcagcagcaacattggttacaactacgtgtactggtaccagcagctgccgggcacggcgccgaaactgctgatctacgaaaacaacaaacgcccgagcggcgtgccggatcgctttagcggatccaaaagcggcaccagcgccagcctggcgattaccggcctgcaagcagaagacgaagcggattattactgctcttcttgggactctcattctaactcttacgtgtttggcggcggcacgaagttaaccgttcttggccagccgaaagccgccccaagcgtgaccctgtttccgccgagcagcgaagaactgcaagccaacaaagccaccctggtttgcctgatcagcgatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

SEQ ID NO: 21 - protein, 1298 aa, Homo sapiens
MKVVPEKNAVRILWGRERGARAMGAQRLLQELVEDKTRWMKWEGKRVELPDSPRSTFLLAFSPDRTLLASTHVNHNIYITEVKTGKCVHSLIGHRRTPWCVTFHPTISGLIASGCLDGEVRIWDLHGGSESWFTDSNNAIASLAFHPTAQLLLIATANEIHFWDWSRREPFAVVKTASEMERVRLVRFDPLGHYLLTAIVNPSNQQGDDEPEIPIDGTELSHYRQRALLQSQPVRRTPLLHNFLHMLSSRSSGIQVGEQSTVQDSATPSPPPPPPQPSTERPRTSAYIRLRQRVSYPTAECCQHLGILCLCSRCSGTRVPSLLPHQDSVPPASARATTPSFSFVQTEPFHPPEQASSTQQDQGLLNRPSAFSTVQSSTAGNTLRNLSLGPTRRSLGGPLSSHPSRYHREIAPGLTGSEWTRTVLSLNSRSEAESMPPPRTSASSVSLLSVLRQQEGGSQASVYTSATEGRGFPASGLATESDGGNGSSQNNSGSIRHELQCDLRRFFLEYDRLQELDQSLSGEAPQTQQAQEMLNNNIESERPGPSHQPTPHSSENNSNLSRGHLNRCRACHNLLTFNNDTLRWERTTPNYSSGEASSSWQVPSSFESVPSSGSQLPPLERTEGQTPSSSRLELSSSASPQEERTVGVAFNQETGHWERIYTQSSRSGTVSQEALHQDMPEESSEEDSLRRRLLESSLISLSRYDGAGSREHPIYPDPARLSPAAYYAQRMIQYLSRRDSIRQRSMRYQQNRLRSSTSSSSSDNQGPSVEGTDLEFEDFEDNGDRSRHRAPRNARMSAPSLGRFVPRRFLLPEYLPYAGIFHERGQPGLATHSSVNRVLAGAVIGDGQSAVASNIANTTYRLQWWDFTKFDLPEISNASVNVLVQNCKIYNDASCDISADGQLLAAFIPSSQRGFPDEGILAVYSLAPHNLGEMLYTKRFGPNAISVSLSPMGRYVMVGLASRRILLHPSTEHMVAQVFRLQQAHGGETSMRRVFNVLYPMPADQRRHVSINSARWLPEPGLGLAYGTNKGDLVICRPEALNSGVEYYWDQLNETVFTVHSNSRSSERPGTSRATWRTDRDMGLMNAIGLQPRNPATSVTSQGTQTLALQLQNAETQTEREVPEPGTAASGPGEGEGSEYGASGEDALSRIQRLMAEGGMTAVVQREQSTTMASMGGFGNNIIVSHRIHRSSQTGTEPGAAHTSSPQPSTSRGLLPEAGQLAERGLSPRTASWDQPGTPGREPTQPTLPSSSPVPIPVSLPSAEGPTLHCELTNNNHLLDGGSSRGDAAGPRGEPRNR

SEQ ID NO: 22 - protein, 19 aa, synthetic construct
  REGION 1..19: note = peptide to which anti-Ambra-1 antibodies were raised
CGGSSRGDAAGPRGEPRNR

SEQ ID NO: 23 - protein, 5 aa, synthetic construct
  REGION 1..5: note = HCDR1 of anti-p62 antibody AbD34907
TYYLH

SEQ ID NO: 24 - protein, 17 aa, synthetic construct
  REGION 1..17: note = HCDR2 of the anti-p62 antibody AbD34907
LINPNNGGTNYAQKFQG

SEQ ID NO: 25 - protein, 14 aa, synthetic construct
  REGION 1..14: note = HCDR3 of the anti-p62 antibody AbD34907
GPGRLRPFGLYFDV

SEQ ID NO: 26 - protein, 11 aa, synthetic construct
  REGION 1..11: note = LCDR1 of the anti-p62 antibody AbD34907
RASQGIYTFLN

SEQ ID NO: 27 - protein, 7 aa, synthetic construct
  REGION 1..7: note = LCDR2 of the anti-p62 antibody AbD34907
AASTLQS

SEQ ID NO: 28 - protein, 9 aa, synthetic construct
  REGION 1..9: note = LCDR3 of the anti-p62 antibody AbD34907
FQYSSWPRT

SEQ ID NO: 29 - protein, 30 aa, synthetic construct
  REGION 1..30: note = HCFR1 of the anti-p62 antibody AbD34907
QVQLVQSGAEVKKPGASVKVSCKASGYTFN

SEQ ID NO: 30 - protein, 14 aa, synthetic construct
  REGION 1..14: note = HCFR2 of the anti-p62 antibody AbD34907
WVRQAPGQGLEWMG

SEQ ID NO: 31 - protein, 32 aa, synthetic construct
  REGION 1..32: note = HCFR3 of the anti-p62 antibody AbD34907
RVTMTRDTSISTAYMELSRLRSEDTAVYYCAR

SEQ ID NO: 32 - protein, 11 aa, synthetic construct
  REGION 1..11: note = HCFR4 of the anti-p62 antibody AbD34907
WGQGTLVTVSS

SEQ ID NO: 33 - protein, 23 aa, synthetic construct
  REGION 1..23: note = LCFR1 of the anti-p62 antibody AbD34907
DIQMTQSPSSLSASVGDRVTITC

SEQ ID NO: 34 - protein, 15 aa, synthetic construct
  REGION 1..15: note = LCFR2 of the anti-p62 antibody AbD34907
WYQQKPGKAPKLLIY

SEQ ID NO: 35 - protein, 32 aa, synthetic construct
  REGION 1..32: note = LCFR3 of the anti-p62 antibody AbD34907
GVPSRFSGSGSGTDFTLTISSLQPEDFATYYC

SEQ ID NO: 36 - protein, 10 aa, synthetic construct
  REGION 1..10: note = LCFR4 of the anti-p62 antibody AbD34907
FGQGTKVEIK

SEQ ID NO: 37 - protein, 123 aa, synthetic construct
  REGION 1..123: note = VH domain of the anti-p62 antibody AbD34907
QVQLVQSGAEVKKPGASVKVSCKASGYTFNTYYLHWVRQAPGQGLEWMGLINPNNGGTNYAQKFQGRVTMTRDTSISTAYMELSRLRSEDTAVYYCARGPGRLRPFGLYFDVWGQGTLVTVSS

SEQ ID NO: 38 - protein, 107 aa, synthetic construct
  REGION 1..107: note = VL domain of the anti-p62 antibody AbD34907
DIQMTQSPSSLSASVGDRVTITCRASQGIYTFLNWYQQKPGKAPKLLIYAASTLQSGVPSRFSGSGSGTDFTLTISSLQPEDFATYYCFQYSSWPRTFGQGTKVEIK

SEQ ID NO: 39 - protein, 225 aa, synthetic construct
  REGION 1..225: note = Fd chain (VH domain and constant domain) of the Fab region of the anti-p62 antibody AbD34907
QVQLVQSGAEVKKPGASVKVSCKASGYTFNTYYLHWVRQAPGQGLEWMGLINPNNGGTNYAQKFQGRVTMTRDTSISTAYMELSRLRSEDTAVYYCARGPGRLRPFGLYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 40 - protein, 214 aa, synthetic construct
  REGION 1..214: note = Light chain (VL domain and constant domain) of the Fab region of the anti-p62 antibody AbD34907
DIQMTQSPSSLSASVGDRVTITCRASQGIYTFLNWYQQKPGKAPKLLIYAASTLQSGVPSRFSGSGSGTDFTLTISSLQPEDFATYYCFQYSSWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 41 - protein, 5 aa, synthetic construct
  REGION 1..5: note = HCDR1 of anti-p62 antibody AbD34908
NYWIG

SEQ ID NO: 42 - protein, 17 aa, synthetic construct
  REGION 1..17: note = HCDR2 of the anti-p62 antibody AbD34908
IIDPSGSFTRYSPSFQG

SEQ ID NO: 43 - protein, 17 aa, synthetic construct
  REGION 1..17: note = HCDR3 of the anti-p62 antibody AbD34908
KYVPPVYSYWSYGTLDV

SEQ ID NO: 44 - protein, 12 aa, synthetic construct
  REGION 1..12: note = LCDR1 of the anti-p62 antibody AbD34908
RASQSVSASRLA

SEQ ID NO: 45 - protein, 7 aa, synthetic construct
  REGION 1..7: note = LCDR2 of the anti-p62 antibody AbD34908
GSSTRAT

SEQ ID NO: 46 - protein, 8 aa, synthetic construct
  REGION 1..8: note = LCDR3 of the anti-p62 antibody AbD34908
QQYLHLYT

SEQ ID NO: 47 - protein, 30 aa, synthetic construct
  REGION 1..30: note = HCFR1 of the anti-p62 antibody AbD34908
EVQLVQSGAEVKKPGESLKISCKGSGYSFT

SEQ ID NO: 48 - protein, 14 aa, synthetic construct
  REGION 1..14: note = HCFR2 of the anti-p62 antibody AbD34908
WVRQMPGKGLEWMG

SEQ ID NO: 49 - protein, 32 aa, synthetic construct; identical to SEQ ID NO: 9
  REGION 1..32: note = HCFR3 of the anti-p62 antibody AbD34908

SEQ ID NO: 50 - protein, 11 aa, synthetic construct
  REGION 1..11: note = HCFR4 of the anti-p62 antibody AbD34908
WGQGTLVTVSS

SEQ ID NO: 51 - protein, 23 aa, synthetic construct
  REGION 1..23: note = LCFR1 of the anti-p62 antibody AbD34908
DIVLTQSPATLSLSPGERATLSC

SEQ ID NO: 52 - protein, 15 aa, synthetic construct
  REGION 1..15: note = LCFR2 of the anti-p62 antibody AbD34908
WYQQKPGQAPRLLIY

SEQ ID NO: 53 - protein, 32 aa, synthetic construct
  REGION 1..32: note = LCFR3 of the anti-p62 antibody AbD34908
GIPARFSGSGSGTDFTLTISSLEPEDFAVYYC

SEQ ID NO: 54 - protein, 10 aa, synthetic construct
  REGION 1..10: note = LCFR4 of the anti-p62 antibody AbD34908
FGQGTKVEIK

SEQ ID NO: 55 - protein, 126 aa, synthetic construct
  REGION 1..126: note = VH domain of the anti-p62 antibody AbD34908
EVQLVQSGAEVKKPGESLKISCKGSGYSFTNYWIGWVRQMPGKGLEWMGIIDPSGSFTRYSPSFQGQVTISADKSISTAYLQWSSLKASDTAMYYCARKYVPPVYSYWSYGTLDVWGQGTLVTVSS

SEQ ID NO: 56 - protein, 107 aa, synthetic construct
  REGION 1..107: note = VL domain of the anti-p62 antibody AbD34908
DIVLTQSPATLSLSPGERATLSCRASQSVSASRLAWYQQKPGQAPRLLIYGSSTRATGIPARFSGSGSGTDFTLTISSLEPEDFAVYYCQQYLHLYTFGQGTKVEIK

SEQ ID NO: 57 - protein, 228 aa, synthetic construct
  REGION 1..228: note = Fd chain (VH domain and constant domain) of the Fab region of the anti-p62 antibody AbD34908
EVQLVQSGAEVKKPGESLKISCKGSGYSFTNYWIGWVRQMPGKGLEWMGIIDPSGSFTRYSPSFQGQVTISADKSISTAYLQWSSLKASDTAMYYCARKYVPPVYSYWSYGTLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 58 - protein, 214 aa, synthetic construct
  REGION 1..214: note = light chain (VL domain and constant domain) of the Fab region of the anti-p62 antibody AbD34908
DIVLTQSPATLSLSPGERATLSCRASQSVSASRLAWYQQKPGQAPRLLIYGSSTRATGIPARFSGSGSGTDFTLTISSLEPEDFAVYYCQQYLHLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 59 - protein, 910 aa, synthetic construct
  REGION 1..910: note = Full amino acid sequence of the anti-p62 antibody AbD34907 including alkaline phosphatase dimerization domain sequence (AP), FLAG and His6 tag
DIQMTQSPSSLSASVGDRVTITCRASQGIYTFLNWYQQKPGKAPKLLIYAASTLQSGVPSRFSGSGSGTDFTLTISSLQPEDFATYYCFQYSSWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEAQVQLVQSGAEVKKPGASVKVSCKASGYTFNTYYLHWVRQAPGQGLEWMGLINPNNGGTNYAQKFQGRVTMTRDTSISTAYMELSRLRSEDTAVYYCARGPGRLRPFGLYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 60 - protein, 913 aa, synthetic construct
  REGION 1..913: note = Full amino acid sequence of the anti-p62 antibody AbD34908 including alkaline phosphatase dimerization domain sequence (AP), FLAG and His6 tag
DIVLTQSPATLSLSPGERATLSCRASQSVSASRLAWYQQKPGQAPRLLIYGSSTRATGIPARFSGSGSGTDFTLTISSLEPEDFAVYYCQQYLHLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEAEVQLVQSGAEVKKPGESLKISCKGSGYSFTNYWIGWVRQMPGKGLEWMGIIDPSGSFTRYSPSFQGQVTISADKSISTAYLQWSSLKASDTAMYYCARKYVPPVYSYWSYGTLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 61 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 62 - protein, 364 aa, synthetic construct
  REGION 1..364: note = p62 protein fragment used to generate anti-p62 antibodies
MAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPLLEHHHHHH

SEQ ID NO: 63 - protein, 41 aa, synthetic construct
  REGION 1..41: note = Ambra-1 C-terminal sequence used for replacement analysis in epitope mapping
VSLPSAEGPTLHCELTNNNHLLDGGSSRGDAAGPRGEPRNR

SEQ ID NO: 64 - protein, 19 aa, synthetic construct
  REGION 1..19: note = REPNET epitope of the Anti-Ambra-1 antibodies
DGGSSRGDAAGPRGEPRNR

SEQ ID NO: 65 - protein, 9 aa, synthetic construct
  REGION 1..9: note = LIN epitope of the Anti-Ambra-1 antibodies
HLLDGGSSR

SEQ ID NO: 66 - protein, 10 aa, synthetic construct
  REGION 1..10: note = LOOP epitope of the Anti-Ambra-1 antibodies
NHLLDGGSSR

SEQ ID NO: 67 - protein, 4 aa, synthetic construct
  REGION 1..4: note = core epitope of the Anti-Ambra-1 antibodies
EPRN

SEQ ID NO: 68 - skipped

SEQ ID NO: 69 - protein, 8 aa, synthetic construct
  REGION 1..8: note = Flag EPITOPE TAG
DYKDDDDK

SEQ ID NO: 70 - protein, 14 aa, synthetic construct
  REGION 1..14: note = V5 EPITOPE TAG
GKPIPNPLLGLDST

SEQ ID NO: 71 - protein, 8 aa, synthetic construct
  REGION 1..8: note = Strep EPITOPE TAG
WSHPQFEK